(19) United States
(12) Patent Application Publication
(10) Pub. No.: US 2011/0130365 A1
(43) Pub. Date: Jun. 2, 2011

(54) Fluorinated Heteroaryls

(76) Inventors: John W. Benbow, Norwich, CT (US); Jihong Lou, San Diego, CA (US); Jeffrey A. Pferfferkorn, Mystic, CT (US); Meihua M. Tu, Mystic, CT (US)

(21) Appl. No.: 13/055,952

(22) PCT Filed: Jul. 15, 2009
(86) PCT No.: PCT/IB09/53068

§ 371 Date: Jan. 26, 2011

Related U.S. Application Data
(60) Provisional application No. 61/084,282, filed on Jul. 29, 2008.
(60) Provisional application No. 61/183,693, filed on Jun. 3, 2009.

(51) Int. Cl. A61K 31/675; C07D 401/12; A61K 31/497; A61K 31/444; A61K 31/4439; A61K 31/506; A61K 31/4709; C07D 403/12; C07F 9/6558; A61P 3/10

(52) U.S. Cl. 514/85; 544/405; 514/255.05; 546/261; 514/335; 546/275.4; 514/341; 544/331; 514/275; 546/272.1; 514/340; 546/159; 514/313; 544/295; 544/337

(57) ABSTRACT

The present invention provides Formula (1A) XN O R 3 HN R 5 O R 4 R 2 R 1 (1A) 5 compounds that act as glucokinase activators; pharmaceutical compositions thereof; and methods of treating diseases, disorders, or conditions mediated by the glucokinase enzyme, where X, R 1, R 2, R 3, R 4, and R 5 are as described herein.

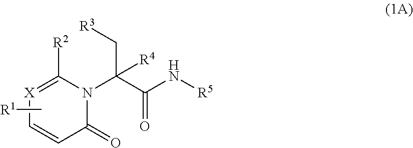

### FIELD OF THE INVENTION

The present invention relates to fluorinated heteroaryls and the uses thereof as glucokinase enzyme activators.

### BACKGROUND

Diabetes is a major public health concern because of its increasing prevalence and associated health risks. The disease is characterized by metabolic defects in the production and utilization of carbohydrates which result in the failure to maintain appropriate blood glucose levels. Two major forms of diabetes are recognized. Type I diabetes, or insulin-dependent diabetes mellitus (IDDM), is the result of an absolute deficiency of insulin. Type II diabetes, or non-insulin dependent diabetes mellitus (NIDDM), often occurs with normal, or even elevated levels of insulin and appears to be the result of the inability of tissues and cells to respond appropriately to insulin. Aggressive control of NIDDM with medication is essential; otherwise it can progress into IDDM.

As blood glucose increases, it is transported into pancreatic beta cells via a glucose transporter. Intracellular mammalian glucokinase is an enzyme that senses the rise in glucose and activates cellular glycolysis, i.e. the conversion of glucose to glucose-6-phosphate, and subsequent insulin release. Glucokinase is found principally in pancreatic β-cells and liver parenchymal cells. Because transfer of glucose from the blood into muscle and fatty tissue is insulin dependent, diabetics lack the ability to utilize glucose adequately which leads to undesired accumulation of blood glucose (hyperglycemia). Chronic hyperglycemia leads to decreases in insulin secretion and contributes to increased insulin resistance. Glucokinase also acts as a sensor in hepatic parenchymal cells which induces glycogen synthesis, thus preventing the release of glucose into the blood. The glucokinase processes are thus critical for the maintenance of whole body glucose homeostasis.

It is expected that an agent that activates cellular glucokinase will facilitate glucose-dependent secretion from pancreatic beta cells, correct postprandial hyperglycemia, increase hepatic glucose utilization and potentially inhibit hepatic glucose release. Consequently, a glucokinase activator may provide therapeutic treatment for NIDDM and associated complications, inter alia, hyperglycemia, dyslipidemia, insulin resistance syndrome, hyperinsulinemia, hypertension, and obesity.

Several drugs in five major categories, each acting by different mechanisms, are available for treating hyperglycemia and subsequently, NIDDM (Moller, D. E., “New drug targets for Type 2 diabetes and the metabolic syndrome” Nature 414; 821-827, (2001)): (A) Insulin secretogogues, including sulphonyl-ureas (e.g., glipizide, glimepiride, glyburide) and meglitinides (e.g., nateglidine and repaglinide) enhance secretion of insulin by acting on the pancreatic beta-cells. While this therapy can decrease blood glucose level, it has limited efficacy and tolerability, causes weight gain and often induces hypoglycemia. (B) Biguanides (e.g., metformin) are thought to act primarily by decreasing hepatic glucose production. Biguanides often cause gastrointestinal disturbances and lactic acidosis, further limiting their use. (C) Inhibitors of alpha-glucosidase (e.g., acarbose) decrease intestinal glucose absorption. These agents often cause gastrointestinal disturbances. (D) Thiazolidinediones (e.g., pioglitazone, rosiglitazone) act on a specific receptor (peroxisome proliferator-activated receptor-gamma) in the liver, muscle and fat tissues. They regulate lipid metabolism subsequently enhancing the response of these tissues to the actions of insulin. Frequent use of these drugs may lead to weight gain and may induce edema and anemia. (E) Insulin is used in more severe cases, either alone or in combination with the above agents.

Ideally, an effective new treatment for NIDDM would meet the following criteria: (a) it would not have significant side effects including induction of hypoglycemia; (b) it would not cause weight gain; (c) it would at least partially replace insulin by acting via mechanism(s) that are independent from the actions of insulin; (d) it would desirably be metabolically stable to allow less frequent usage; and (e) it would be usable in combination with tolerable amounts of any of the categories of drugs listed herein.

Substituted heteroaryls, particularly pyridones, have been implicated in mediating GK and may play a significant role in the treatment of NIDDM. For example, U.S. Patent publication No. 2006/0058353 and PCT publication No's. WO2007/043638, WO2007/043638, and WO2007/117995 recite certain heterocyclic derivatives with utility for the treatment of diabetes. Although investigations are on-going, there still exists a need for a more effective and safe therapeutic treatment for diabetes, particularly NIDDM.

### SUMMARY

The present invention provides Formula (1A) compounds that act as glucokinase modulators, in particular, glucokinase activators,

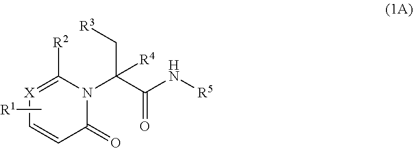

X is carbon or nitrogen;

R¹ is —CF₂R^{a} where R^{a} is H, F, or (C₁-C₆)alkyl;

R² is H, halo, CF₃, (C₁-C₆)alkyl, or (C₁-C₃)alkoxy;

R³ is a chemical moiety selected from the group consisting of (C₃-C₆)cycloalkyl, 5- to 6-membered heterocycle, 5- to 6-membered heteroaryl, and phenyl, wherein said heterocycle or said heteroaryl contains one to three heteroatoms each independently N, O, or S, and where said moiety is optionally substituted with one to three substituents each independently halo, (C₁-C₆)alkyl, (C₁-C₆)alkoxy, CF₃, or cyano;

R⁴ is H or (C₁-C₆)alkyl; and

R⁵ is a chemical moiety selected from the group consisting of a 5- to 6-membered heteroaryl and quinolinyl, wherein said heteroaryl contains one to three heteroatoms each independently N, O, or S, and where said moiety is optionally substituted with one to three R⁶ substituents each independently (C₁-C₆)alkyl, CF₃, cyano, (C₁-C₆)alkoxy, halo, amino, (C₁-C₃)alkylamino, di-(C₁-C₃)alkylamino, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, and where the aryl of said arylalkyl is optionally substituted with one to three substituents each independently (C₁-C₆)alkyl, CF₃, cyano, (C₁-C₆)alkoxy, halo, carboxy, amino, (C₁-C₃)alkylamino, or di-(C₁-C₃)alkylamino;

or a pharmaceutically acceptable salt thereof.

Preferrably, R¹ is —CHF₂, —CF₃, —CF₂CH₃, —CF₂CH₂CH₃, or —CF₂CH(CH₃)₂. More preferred, R¹ is —CHF₂, —CF₃, —CF₂CH₃, or —CF₂CH₂CH₃. Most preferred, R¹ is —CHF₂, —CF₃, —CF₂CH₃, or —CF₂CH₂CH₃.

Preferrably, R² is H, F, Cl, CF₃, methyl, ethyl, propyl, isopropyl, butyl, n-butyl, methoxy, or ethoxy. More preferred, R² is H, F, Cl, CF₃, methyl, ethyl, methoxy, or ethoxy. Most preferred, R² is H.

Preferrably, R³ is a chemical moiety selected from the group consisting of cyclobutyl, cyclopentyl, cyclohexyl, tetrahydrofuranyl, tetrahydrothiophenyl, pyrrolidinyl, tetrahydropyranyl, and piperidinyl, where said moiety is optionally substituted with one to three optional substituents. Preferred R³ substituents are independently halo, methyl, ethyl, methoxy, alkoxy, or cyano. More preferred, R³ is a chemical moiety selected from the group consisting of cyclobutyl, cyclopentyl, cyclohexyl, tetrahydrofuranyl, tetrahydropyranyl, and piperidinyl, where said moiety is optionally substituted with one substituent. When substituted, the more preferred R³ substituent is halo, methyl, methoxy, or cyano. Most preferred, R³ is cyclopentyl or tetrahydropyranyl.

Preferrably, R⁴ is H, methyl, ethyl, propyl, or isopropyl. More preferred, R⁴ is H, methyl or ethyl. Most preferred, R⁴ is H.

Preferrably, R⁵ is a chemical moiety selected from the group consisting of pyrrolyl, furanyl, pyrazolyl, imidazolyl, isoxazolyl, oxazolyl, isothiazolyl, thiazolyl, pyridinyl, pyridazinyl, pyrimidinyl, pyrazinyl, and quinolinyl, where said moiety is optionally substituted with one to three R⁶ substituents independently (C₁-C₆)alkyl, CF₃, cyano, (C₁-C₆)alkoxy, halo, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, where the aryl of said arylalkyl is optionally substituted with one to three substituents each independently methyl, ethyl, CF₃, cyano, methoxy, ethoxy, F, Cl, or carboxy. More preferred, R⁵ is a chemical moiety selected from the group consisting of pyrazolyl, isoxazolyl, thiazolyl, pyridinyl, pyrimidinyl, pyrazinyl, and quinolinyl, where said moiety is optionally substituted with one R⁶ substituent. More preferred, R⁶ is (C₁-C₆)alkyl, CF₃, (C₁-C₆)alkoxy, halo, —CH₂P(O)(OR⁷)(OR⁸), —C(O)OR⁷, —CH₂C(O)OR⁷, or benzyl, where R⁷ and R⁸ are each independently H or (C₁-C₆)alkyl. Most preferred, R⁵ is a chemical moiety selected from the group consisting of Formula's (a), (b), (c), (d), (e), (f), (g), (h), (i), (j), (k), and (l)

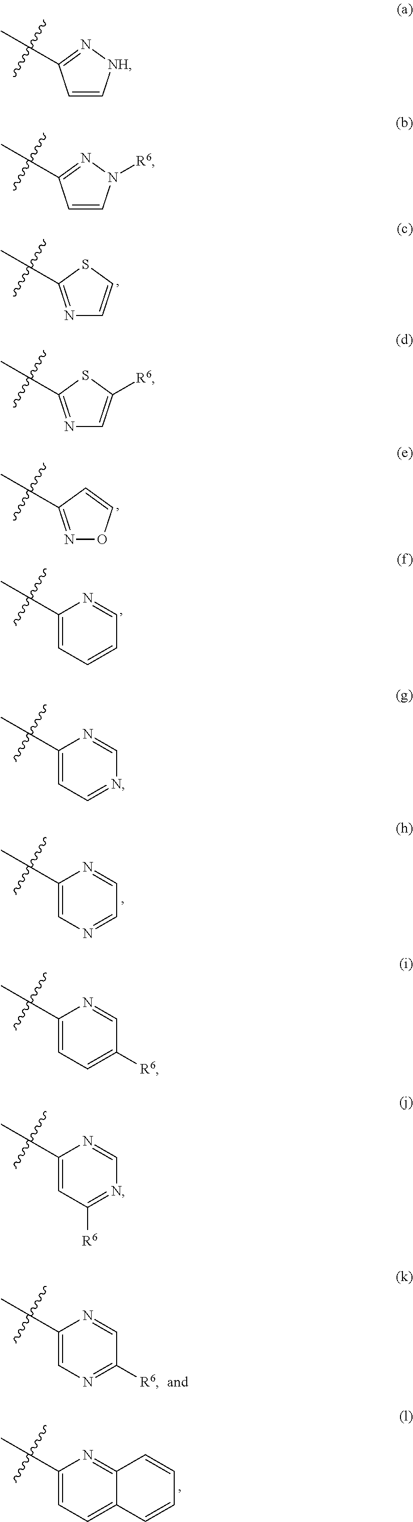

where [symbol] is point of attachment, and where R⁶ is methyl, ethyl, methoxy, CF₃, methoxy, ethoxy, halo, —CH₂P(O)(OR⁷)(OR⁸), —C(O)OR⁷, —CH₂C(O)OR⁷, or benzyl, wherein R⁷ and R⁸ are each independently H, methyl, or ethyl.

When X is carbon, preferred Formula (1A) compounds are selected from the group consisting of Formulas (1B) and (1C),

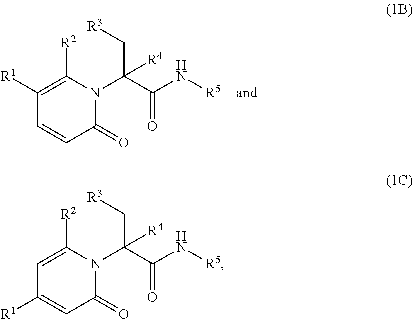

where R¹, R², R³, R⁴, and R⁵ are as described above. More preferred, are Formula (1B) compounds.

Preferred Formula (1B) and (1C) compounds include:
(S)-3-cyclopentyl-N-(5-methylpyridin-2-yl)-2-(2-oxo-4-(trifluoromethyl)pyridin-1(2H)-yl)propanamide;
(S)-3-cyclopentyl-N-(5-methylpyridin-2-yl)-2-(2-oxo-5-(trifluoromethyl)pyridin-1(2H)-yl)propanamide;
(S)-3-cyclopentyl-N-(5-methylpyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyrazin-2-yl)propanamide;
(S)-3-cyclopentyl-2-(2-oxo-5-(trifluoromethyl)pyridin-1(2H)-yl)-N-(pyrimidin-4-yl)propanamide;
(S)-3-cyclopentyl-N-(1-ethyl-1H-pyrazol-3-yl)-2-(2-oxo-5-(trifluoromethyl)pyridin-1(2H)-yl)propanamide;
(S)—N-(1-benzyl-1H-pyrazol-3-yl)-3-cyclopentyl-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyrimidin-2-yl)propanamide;
(S)-3-cyclopentyl-N-(1-methyl-1H-pyrazol-3-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(5-(trifluoromethyl)pyridin-2-yl)propanamide;
(S)-3-cyclopentyl-2-(2-oxo-5-(trifluoromethyl)pyridin-1(2H)-yl)-N-(quinolin-2-yl)propanamide;
(S)-3-cyclopentyl-N-(5-methoxypyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyridin-2-yl)propanamide;
(S)-2-(2-(3-cyclopentyl-2-(2-oxo-5-(trifluoromethyl)pyridin-1(2H)-yl)propanamido)thiazol-5-yl)acetic acid monoacetate;
(S)-methyl 6-(3-cyclopentyl-2-(2-oxo-5-(trifluoromethyl)pyridin-1(2H)-yl)propanamido)nicotinate;
(S)-6-(3-cyclopentyl-2-(2-oxo-5-(trifluoromethyl)pyridin-1(2H)-yl)propanamido)nicotinic acid mono acetate; and
(S)-diethyl(5-(3-cyclopentyl-2-(2-oxo-5-(trifluoromethyl)pyridin-1(2H)-yl)propanamido)-pyrazin-2-yl)methylphosphonate, or a pharmaceutically acceptable salt thereof.

More preferred compounds include:
(S)-3-cyclopentyl-N-(5-methylpyridin-2-yl)-2-(2-oxo-4-(trifluoromethyl)pyridin-1(2H)-yl)propanamide;
(S)-3-cyclopentyl-N-(5-methylpyridin-2-yl)-2-(2-oxo-5-(trifluoromethyl)pyridin-1(2H)-yl)propanamide;
(S)-3-cyclopentyl-N-(5-methylpyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyrimidin-4-yl)propanamide;
(S)—N-(1-benzyl-1H-pyrazol-3-yl)-3-cyclopentyl-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(5-(trifluoromethyl)pyridin-2-yl)propanamide;
(S)-3-cyclopentyl-2-(2-oxo-5-(trifluoromethyl)pyridin-1(2H)-yl)-N-(quinolin-2-yl)propanamide;
(S)-3-cyclopentyl-N-(5-methoxypyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyridin-2-yl)propanamide;
(S)-methyl 6-(3-cyclopentyl-2-(2-oxo-5-(trifluoromethyl)pyridin-1(2H)-yl)propanamido)nicotinate; and
(S)-6-(3-cyclopentyl-2-(2-oxo-5-(trifluoromethyl)pyridin-1(2H)-yl)propanamido)nicotinic acid mono acetate;
   or a pharmaceutically acceptable salt thereof.

Most preferred compounds include:
(S)-3-cyclopentyl-N-(5-methylpyridin-2-yl)-2-(2-oxo-5-(trifluoromethyl)pyridin-1(2H)-yl)propanamide;
(S)—N-(1-benzyl-1H-pyrazol-3-yl)-3-cyclopentyl-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(5-(trifluoromethyl)pyridin-2-yl)propanamide;
(S)-3-cyclopentyl-2-(2-oxo-5-(trifluoromethyl)pyridin-1(2H)-yl)-N-(quinolin-2-yl)propanamide;
(S)-3-cyclopentyl-2-(2-oxo-5-(trifluoromethyl)pyridin-1(2H)-yl)-N-(pyridin-2-yl)propanamide;
(S)-methyl 6-(3-cyclopentyl-2-(2-oxo-5-(trifluoromethyl)pyridin-1(2H)-yl)propanamido)nicotinate; and
(S)-6-(3-cyclopentyl-2-(2-oxo-5-(trifluoromethyl)pyridin-1(2H)-yl)propanamido)nicotinic acid mono acetate;
   or a pharmaceutically acceptable salt thereof.

When X is nitrogen, preferred Formula (1A) compounds are selected from the group consisting of Formulas (1D) and (1E),

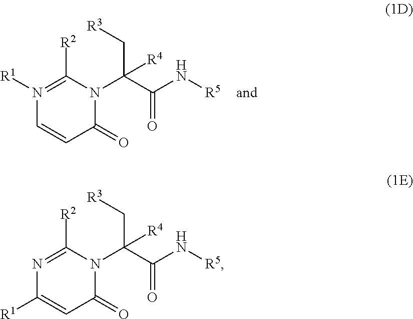

where R¹, R², R³, R⁴, and R⁵ are as described above. A preferred Formula (1E) compound is (S)-3-cyclopentyl-N-(5-methylpyridin-2-yl)-2-(6-oxo-4-(trifluoromethyl)pyrimidin-1(6H)-yl)propanamide; or a pharmaceutically acceptable salt thereof.

Other preferred compounds of the invention include (S)-3-cyclopentyl-N-(5-(hydroxymethyl)pyridin-2-yl)-2-(2-oxo-5-(trifluoromethyl)pyridin-1(2H)-yl)propanamide; (S)-6-(3-cyclopentyl-2-(2-oxo-4-(trifluoromethyl)pyridin-1(2H)-yl)propanamido)nicotinic acid; and (S)-6-(3-cyclohexyl-2-(2-oxo-4-(trifluoromethyl)pyridin-1(2H)-yl)propanamido) nicotinic acid; or a pharmaceutically acceptable salt thereof.

Another embodiment of the present invention is a pharmaceutical composition that comprises (a) a Formula (1A) compound, or a pharmaceutically acceptable salt thereof; and (b) a pharmaceutically acceptable excipient, diluent, or carrier. Preferrably, the composition comprises a therapeutically effective amount of a compound of the present invention, or a pharmaceutically acceptable salt thereof.

The composition may comprise at least one additional pharmaceutical agent, or a pharmaceutically acceptable salt thereof. Preferred additional pharmaceutical agents include anti-diabetes, anti-obesity, anti-hypertension, anti-hyperglycemic, and lipid lowering agents, as described herein. More preferred, are anti-diabetic and anti-obesity agents, as described herein.

In yet another embodiment of the present invention is a method for treating a disease, condition, or disorder mediated by the activation of glucokinase in a mammal that includes the step of administering to a mammal, preferably a human, in need of such treatment, a therapeutically effective amount of a compound of the present invention, or a pharmaceutical composition thereof.

Diseases, disorders, or conditions mediated by glucokinase activators include Type II diabetes, hyperglycemia, metabolic syndrome, impaired glucose tolerance, glucosuria, cataracts, diabetic neuropathy, diabetic nephropathy, diabetic retinopathy, obesity, dyslididemia, hypertension, hyperinsulinemia, and insulin resistance syndrome. Preferred diseases, disorders, or conditions include Type II diabetes, hyperglycemia, impaired glucose tolerance, obesity, and insulin resistance syndrome. More preferred are Type II diabetes, hyperglycemia, and obesity. Most preferred is Type II diabetes.

One aspect of the present invention is a method of reducing the level of blood glucose in a mammal, preferably a human, which includes the step of administering to a mammal in need of such treatment a therapeutically effective amount of a compound of the present invention, or a pharmaceutical composition thereof.

Compounds of the present invention may be administered in combination with other pharmaceutical agents (in particular, anti-obesity and anti-diabetic agents described herein). The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### DEFINITIONS

For purposes of the present invention, as described and claimed herein, the following terms and phrases are defined as follows:

“Activate(s)” or “activator”, or “activation”, as used herein, unless otherwise indicated, refers to the ability of the compounds of the present invention to indirectly or directly bind to the glucokinase enzyme in a mammal as a ligand thereby partially or wholly activating said enzyme.

“Additional pharmaceutical agent(s)” as used herein, unless otherwise indicated, refers to other pharmaceutical compounds or products that provide a therapeutically effective amount of said agents that are useful for the treatment of a disease, condition, or disorder, as described herein.

“Alkoxy”, as used herein, unless otherwise indicated, refers to an oxygen moiety having a further alkyl substituent. The alkyl portion (i.e., alkyl moiety) of an alkoxy group has the same definition as below.

“Alkyl”, as used herein, unless otherwise indicated, includes saturated monovalent hydrocarbon alkane radicals of the general formula CₙH₂ₙ₊₁. The alkane radical may be straight or branched and may be unsubstituted or substituted. For example, the term “(C₁-C₆) alkyl” refers to a monovalent, straight or branched aliphatic group containing 1 to 6 carbon atoms. Non-exclusive examples of (C₁-C₆) alkyl groups include, but are not limited to methyl, ethyl, propyl, isopropyl, sec-butyl, t-butyl, n-propyl, n-butyl, i-butyl, s-butyl, n-pentyl, 1-methylbutyl, 2-methylbutyl, 3-methylbutyl, neopentyl, 3,3-dimethylpropyl, 2-methylpentyl, hexyl, and the like. Alkyl represented along with another term (e.g., alkylamino (e.g., CH₃NH—), aminoalkyl (e.g., NH₂CH₂—), di-alkylamino (e.g., (CH₃)₂N—), arylalkyl (e.g., benzyl), and the like) where said alkyl moiety has the same meaning as above and may be attached to the chemical moiety by any one of the carbon atoms of the aliphatic chain.

“Aryl”, as used herein, unless otherwise indicated, refers to a monocyclic, bicyclic, or fused ring system wherein each ring is aromatic. A typical aryl group (e.g. phenyl, naphthyl) is a 6- to 10-membered carbocyclic ring or ring system. The aryl group may be attached to the chemical moiety by any one of the carbon atoms within the ring system. Aryl rings are optionally substituted with one to three substituents.

“Compounds of the present invention”, as used herein, unless otherwise indicated, refers to compounds of Formula (1A), pharmaceutically acceptable salts of the compounds, as well as, all stereoisomers (e.g., enantiomers), tautomers and isotopically labeled compounds, and are therefore considered equivalents of the compounds of the present invention. Solvates and hydrates of the Formula 1A compounds, or a pharmaceutically acceptable salt thereof, are considered compositions.

“Cycloalkyl”, as used herein, unless otherwise indicated, includes fully saturated or partially saturated carbocyclic alkyl moieties, wherein alkyl is as defined above. Preferred cycloalkyls are 3- to 6-membered monocyclic rings including cyclopropyl, cyclobutyl, cyclopentyl, and cyclohexyl. The cycloalkyl group may be attached to the chemical moiety by any one of the carbon atoms within the carbocyclic ring. Cycloalkyl groups are optionally substituted with one to three substituents.

“Diabetes”, as used herein, unless otherwise indicated, refers to metabolic defects in the production and utilization of carbohydrates, particularly glucose, which result in the failure of glucose homeostasis. Preferred forms of diabetes include Type I diabetes, or insulin-dependent diabetes mellitus (IDDM) which results from the absolute deficiency of insulin and Type II diabetes, or non-insulin dependent diabetes mellitus (NIDDM), which often occurs with normal, or even elevated levels of insulin and appears to be the result of the inability of mammalian cells and tissues to respond appropriately to insulin. Most preferred is NIDDM.

“Diabetes-related disorder”, as used herein, unless otherwise indicated, refers to metabolic syndrome (Syndrome X, or elevated blood glucose, hypertension, obesity, dyslipidemia), hyperglycemia, hyperinsulinemia, impaired glucose tolerance, impaired fasting glucose, insulin resistance, obesity, atherosclerotic disease, cardiovascular disease, cerebrovascular disease, peripheral vessel disease, lupus, polycystic ovary syndrome, carcinogenesis, diabetic neuropathy, diabetic nephropathy, diabetic retinopathy, diabetic macular edema, and hyperplasia.

“Heteroaryl”, as used herein, unless otherwise indicated, refers to an aromatic monocyclic ring containing one or more heteroatoms each independently selected from N, O, or S, preferably from one to three heteroatoms. Non-exclusive examples of monocyclic rings include pyrolyl, furanyl, thiophenyl, pyrazolyl, oxazolyl, thiazolyl, oxadiazolyl, pyridinyl, tetrazolyl, pyridazinyl, pyrimidinyl, and the like. The heteroaryl group may be attached to the chemical moiety by any one of the carbon atoms or heteroatoms (e.g., N, O, and S) within the ring. Heteroaryls are optionally substituted with one to three substituents.

“Heterocycle”, as used herein, unless otherwise indicated, refers to non-aromatic rings containing one or more heteroatoms each independently selected from N, O, or S, preferably from one to three heteroatoms, that are either partially saturated or fully saturated and exist as a monocyclic ring. Non-exclusive examples of monocyclic heterocycles include: tetrahydrofuranyl, pyrrolidinyl, tetrahydropyranyl, tetrahydrothiophenyl, piperidinyl, piperazinyl, azathianyl, and the like. The heterocyclic group may be attached to the chemical moiety by any one of the carbon atoms or heteroatoms (e.g. N, O, and S) within the ring. Heterocycles are optionally substituted with one to three substituents.

“Mammal”, or “mammalian” as used herein, unless otherwise indicated, refers to an individual animal that is a member of the taxonomic class Mammalia. Non-exclusive examples of mammals include humans, dogs, cats, horses, and cattle, preferably human.

“Mediate(s)” or “mediated”, as used herein, unless otherwise indicated, refers to the activation of the glucokinase enzyme by enhancing glucose binding, alleviating the inhibition of glucokinase regulatory protein, a key regulator of glucokinase activity in the liver, and/or to increase the catalytic rate of the glucokinase enzyme (e.g., change Vmax).

“Obesity” and “obese”, as used herein, unless otherwise indicated, refers generally to individuals who are at least about 20-30% over the average weight for his/her age, sex and height. Technically, obese is defined, for males and females, as individuals whose body mass index is greater than 27.8 kg/m², and 27.3 kg/m², respectively. Those of skill in the art readily recognize that the invention method is not limited to those who fall within the above criteria. Indeed, the method of the invention can also be advantageously practiced by individuals who fall outside of these traditional criteria, for example, by those who may be prone to obesity.

“Pharmaceutically acceptable” as used herein, unless otherwise indicated, indicates that the substance or composition must be compatible chemically and/or toxicologically, with the other ingredients comprising a formulation, composition, and/or the mammal being treated therewith.

“Reducing the level of blood glucose”, or “lower blood glucose” as used herein, unless otherwise indicated, refers to an amount of the compound of the present invention sufficient to provide circulating concentrations of the compound high enough to accomplish the desired effect of lowering blood glucose levels in a mammal.

“Therapeutically effective amount”, as used herein, unless otherwise indicated, refers to an amount of the compounds of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

“Treatment”, “treating”, and the like, as used herein, unless otherwise indicated, refers to reversing, alleviating, or inhibiting the progress of the disorder or condition to which such term applies, or one or more symptoms of such disorder or condition. As used herein, these terms also encompass, depending on the condition of the mammal, preferably a human, preventing the onset of a disorder or condition, or of symptoms associated with a disorder or condition, including reducing the severity of a disorder or condition or symptoms associated therewith prior to affliction with said disorder or condition. Thus, treatment can refer to administration of the compounds of the present invention to a mammal that is not at the time of administration afflicted with the disorder or condition. Treating also encompasses preventing the recurrence of a disorder or condition or of symptoms associated therewith.

### DETAILED DESCRIPTION OF THE INVENTION

The present invention provides Formula (1A) compounds, or pharmaceutically acceptable salts thereof, compositions and pharmaceutical compositions that are useful in the treatment of diseases, disorders, or conditions mediated by glucokinase activation, in particular, compounds that activate the glucokinase enzyme in a mammal, preferably a human.

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, “Reagents for Organic Synthesis”, 1; 19, Wiley, New York (1967, 1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below demonstrate potential routes for synthesizing compounds of the present invention, and key intermediates. For a more detailed description of the individual reaction steps, see the Examples section below. Those skilled in the art will appreciate that other suitable starting materials, reagents, and synthetic routes may be used to synthesize the compounds of the present invention and a variety of derivatives thereof. Further, many of the compounds prepared by the methods described below can be further modified in light of this disclosure using conventional chemistry well known to those skilled in the art.

Compounds of the present invention described herein contain at least one asymmetric or chiral center; and therefore, exist in different stereoisomeric forms. The R and S configurations are based upon knowledge of known chiral inversion/retention chemistry by those skilled in the art. For example, the chirality of intermediate undergoes an inversion when a neucleophile attacks from the opposite side of the leaving group, the product could be designated as R or S depending on the priorities of the groups attached to the stereocenter (Stereochemistry of Organic Compounds, by Ernest L. Eliel, Samuel H. Wilen, John Wiley and Sons, Inc. (1994)). Whereas, if a neucleophile attaches to the same side as the leaving group the chirality of intermediate is retained. In most of the examples, there is an inversion of the configuration where a compound with R configuration is converted to compound with a S configuration as the priorities of the all the four substituents at the stereocenter is retained. It is further noted that the intermediates can also be racemic (50:50 mixture of stereoisomer) thereby producing racemic products. A chiral separation method can be used to separate these enantiomers to provide the specific R or S isomers. It is further noted that the intermediates can also be racemic thereby producing racemic products. A more detailed description of techniques that can be used to resolve stereoisomers of compounds from their racemic mixture can be found in Jean Jacques Andre Collet, Samuel H. Wilen, Enantiomers, Racemates and Resolutions, John Wiley and Sons, Inc. (1981). In addition, the present invention embraces all geometric and positional isomers. For example, if a compound of the present invention incorporates a double bond or a fused ring, both the cis- and trans-forms, as well as mixtures, are embraced within the scope of the invention.

In the preparation of compounds of the present invention, protection of remote functionality (e.g., primary or secondary amine) of intermediates from undesired reactions with a protecting or blocking group. The term “protecting group” or “Pg” refers to a substituent that is commonly employed to block or protect a particular functionality while reacting other functional groups on the compound. For example, an amino-, hydroxyl-, or carboxy-protecting group is a substituent attached to an amino-, hydroxyl-, or carboxy-group that blocks or protects the amino-, hydroxyl-, or carboxy-functionality, respectively, in the compound. Suitable amino protecting groups include 1-t-butyloxycarbonyl, acyl groups (e.g., formyl, acetyl, chloroacetyl, trichloroacetyl, trifluoracetyl, o-nitrophenylacetyl, o-nitrophenoxyacetyl, trifluoroacetyl, 4-chlorobutyryl, isobutyryl, o-nitrocinnamoyl, picolinoyl, acylisothiocyanate, aminocaproyl, and benzoyl), and acyloxy groups (e.g., methoxycarbonyl, 9-fluorenylmethoxycarbonyl (Fmoc), 2,2,2-trifluoroethoxycarbonyl, 2-trimethylsilylethxoycarbonyl, vinyloxycarbonyl, allyloxycarbonyl, 1,1-dimethylpropynyloxycarbonyl, benzyloxycarbonyl (CBZ), p-nitrobenzyloxycarbony, and 2,4-dichlorobenzyloxycarbonyl). Suitable hydroxyl protecting groups include acetyl, trimethylsilyl, triethylsilyl, t-butyldimethylsilyl and silyl. Suitable carboxy protecting groups include—methyl-, ethyl-, and t-butyl-esters, trimethylsilyl (TMS), t-butyldimethylsilyl (TBS), diphenylmethyl (benzhydryl, DPM), cyanoethyl, 2-(trimethylsilyl)ethyl, nitroethyl, 2-(trimethylsilyl)ethoxymethyl, 2-(p-toluenesulfonyl)ethyl, and the like. Suitable protecting groups and their respective use are readily determined by one skilled in the art. For a general description of protecting groups and their use, see T. W. Greene, Protective Groups in Organic Synthesis, John Wiley & Sons, New York, 1991.

The term “leaving group” or “L”, as used herein, refers to the group with the meaning conventionally associated with it in synthetic organic chemistry, i.e., an atom or group displaceable under reaction (e.g., alkylating) conditions. Examples of leaving groups include halo (e.g., Cl, F, Br, I), alkyl (e.g., methyl and ethyl), thiomethyl, tosylates, mesylates, and the like. Preferably, the leaving group is a triflate or iodo group.

Schemes 1 through 4 outline the general procedures useful for the preparation of compounds of the present invention. It is to be understood, however, that the invention, as fully described herein and as recited in the claims, is not intended to be limited by the details of the following schemes or modes of preparation.

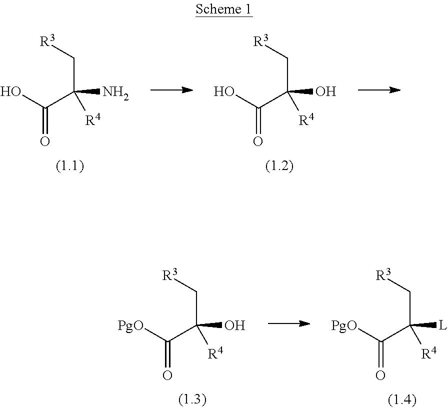

The activated ester (1.4) for introduction of the N-linked heterocycle in the a-position of the ester can be synthesized via treatment with an activating agent such trifluoromethanesulfonic anhydride from the corresponding alcohol (1.3) (Degerbeck, F., et. al., J. Chem. Soc., Perkin Trans. 1, 11-14, (1993)). The a-hydroxy-ester can be prepared from corresponding amino acid (1.1) (McCubbin, J. A., et. al., Org. Letters, 8, 2993-2996, (2006)). The starting amino acids can be purchased from Fulcrum Scientific Limited (West Yorkshire, UK), Sigma-Aldrich (St. Louis, Mo.), and Amatek Chemical (Kowloon, Hong Kong).

As described in Scheme 1, above, “Pg” represents a carboxylic acid protecting group suitable for preventing undesired reactions at a carboxyl group. Representative carboxyl protecting groups include, but are not limited to, esters, such as methyl, ethyl, tert-butyl, benzyl, p-methoxybenzyl, trimethylsilyl (TMS), tert-butyldimethylsilyl (TBS), diphenylmethyl (DPM) and the like. The letter “L” refers to a leaving group which undergoes nucleophilic substitution with a nucleophile. L may refer to any halogen (e.g., chlorine, bromine, fluorine, or iodine), triflate, mesylate, or tosylate. Preferably, L is a triflate or iodo group. It is noted that the intermediates may be synthesized by other reagents known to those skilled in the art.

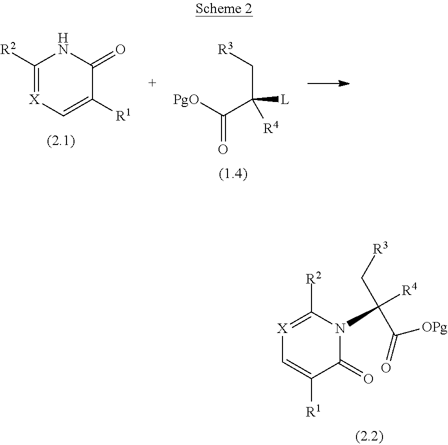

Scheme 2 describes the preparation of a carboxy-protected 2-heterocycle-substituted-ester (2.2). In general, a suitably substituted acid derivative for nucleophilic substitution at the α-position can be achieved by using a leaving group (“L”) which undergoes nucleophilic substitution with a nucleophile with an inversion of configuration. The nucleophile is generated by treatment of (2.1) with NaH or lithium bis(trimethylsilyl)amide and subsequent addition of (1.4) thereby generating intermediate (2.2). Golec, J. M. C., et. al., Bioorg. Med. Chem. Lett., 7, Issue 17, 2181-2186, (1997). It is noted that other bases with an appropriate pK_{b} can also be used for the reaction. The hetero intermediate 2.1 can be purchased from Matrix Scientific (Columbia, S.C.) or TCI America Organic Chemicals (Portland, Oreg.), for example, 4-(trifluoromethyl)pyridin-2(1H)-one, 3-(trifluoromethyl)pyridin-2(1H)-one, 6-(trifluoromethyl)pyrimidin-4(3H)-one, or it can be prepared according to procedures reported in EP408196, for example, 5-(trifluoromethyl)pyrazin-2(1H)-one. The compounds of the present invention are not limited to these hetero intermediates, other intermediates can be used. For example, substituted pyridine-2(1H)-one, substituted pyrazin-2(1H)-one, or a substituted 6-(trifluoromethyl)pyrimidin-4(3H)-one can be used.

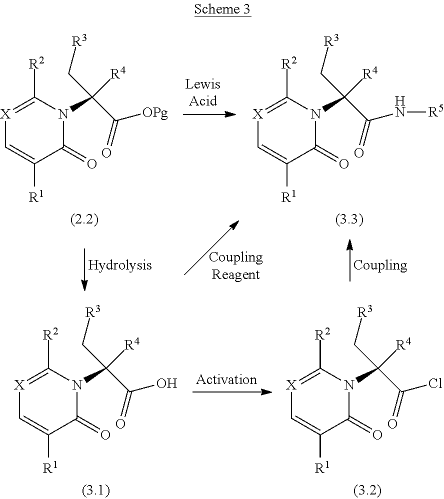

The final transformation to the pyridone amide can be accomplished with a Lewis acid, also referred to as an aprotic acid, and a catalyzed transamidation reaction. For example, transformation of the hetero-substituted ester (2.2) to the amide (3.3) can be achieved by treatment with AlMe₃ or AlMe₂Cl. Yadav, J. S., et. al., Tet. Letters, 48, Issue 24, 4169-4172, (1977). Other suitable Lewis acids include Al₂O₃, TiO₂, ZnCl₂, SnCl₄, TiCl₄, FeCl₃, Be₃F₃, and the like. Alternatively, this transformation can be achieved via hydrolysis of the ester (2.2) to the corresponding carboxylic acid (3.1) followed by coupling with an appropriate amine in presence of a coupling reagent to produce the pyridone amide (i.e., a compound of the present invention). The term “coupling reagent” refers to a chemical reagent that is commonly employed as an agent to couple or join two or more specific compounds to make a single combined compound. Suitable coupling agents include [O-(7-azabenzotriazol-1-yl)-N,N,N′,N′-tetramethyluronium hexafluorophosphate], 1,1′-thiocarbonyldimidazole, and the like. The hydrolysis of the ester can be performed under either basic or acidic conditions. For example, aqueous NaOH, KOH, or LiOH in the presence of an inert organic solvent such as THF or dioxane can be used for base catalyzed hydrolysis. For acid catalyzed hydrolysis, HCl in the presence of water with or without an organic solvent can be used. See, e.g., Puschl, A., et. al., J. Chem. Soc., Perkin Transactions 1, (21), 2757-2763, (2001). Other suitable methods can be used to catalyze the hydrolysis. The carboxylic acid can also be converted to the corresponding acid chloride (3.2), preferably by treatment with oxalyl chloride in presence of catalytic amount of DMF. However, the preparation of the acid chloride is not limited to this reagent only. The acid chloride (3.2) then can be coupled with an appropriate amine to provide the desired amide (3.3). The carboxylic acid can be converted to the corresponding acid chloride which can be converted to the desired amide (3.3). The term “coupling reagent” refers to a chemical reagent that is commonly employed as an agent to couple or join two or more specific compounds to make a single combined compound. Suitable coupling agents include [O-(7-azabenzotriazol-1-yl)-N,N,N′,N′-tetramethyluronium hexafluorophosphate], 1,1′-thiocarbonyldimidazole, and the like.

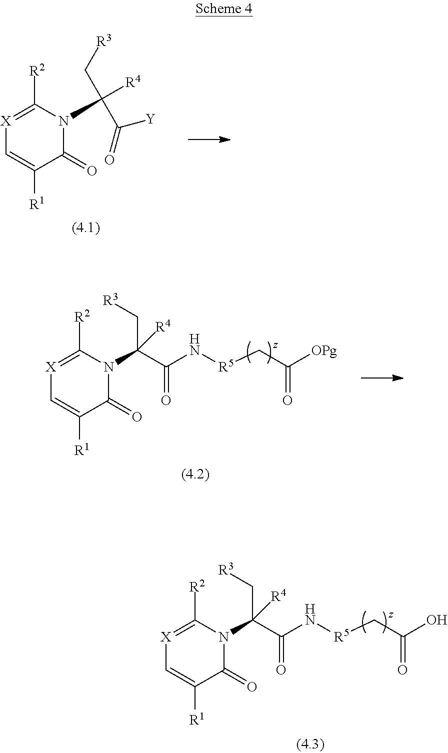

The intermediate amide (4.2) can be prepared from the corresponding acid or acid chloride (4.1) as described in general Schemes 3. For compounds of Formula (4.1), Y is OH or Cl. In a preferred situation, the amide (4.2) can be synthesized by coupling the acid chloride (4.1, Y=Cl) with an appropriate amine. The preparation of the acid (4.1, Y=OH) and the acid chloride and there conversion to the amide were discussed above in Scheme 3. Acid or base catalyzed hydrolysis of ester (4.1) can be utilized to generate the desired acid (4.2). In preferred examples, z is 0 or 1, where the carboxylic acid moiety is separated by none or one methylene linker. Acid or base catalyzed hydrolysis of ester (4.1) can be utilized to generate the desired acid (4.2). Methods for acid or base hydrolysis of the ester are as described in Scheme 3, above.

Compounds of the present invention may be isolated and used per se or optionally administered in the form of its pharmaceutically acceptable salts, hydrates, and/or solvates. For example, it is well within the scope of the present invention to convert the compounds of the present invention into and use them in the form of their pharmaceutically acceptable salts derived from various organic and inorganic acids and bases, acids of amino acids, salts derived form organic and inorganic acids and cationic salts based on the alkali and alkaline earth metals in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydrofluoride, hydroiodide; other mineral acids and their corresponding salts such as sulfate, nitrate, phosphate; and alkyl and monoarysulfonates such as ethanesulfonate, toluenesulfonate, and benzene sulfonate; and other organic acids and their corresponding salts such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include sulfate, pyrosulfate, bisulfate, sulfite, bisulfite, nitrate, phosphate, monohydrogenphosphate, dihydrogenphosphate, metaphosphate, pyrophosphate, trifluoroacetate, propionate, caprylate, isobutyrate, oxalate, malonate, succinate, suberate, sebacate, fumarate, acetate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate, gluconate, galacturonate, and the like. See, e.g., Berge S. M., et. al., Pharmaceutical Salts, J. of Pharma. Sci., 66:1 (1977).

Compounds of the present invention that comprise basic nitrogen-containing groups may be quaternized with such agents as (C₁-C₄)alkyl halides, e.g., methyl, ethyl, isopropyl, and tert-butyl chlorides, bromides, and iodides; di-(C₁-C₄)alkyl sulfates, e.g., dimethyl, diethyl, and diamyl sulfates; (C₁₀-C₁₈)alkyl halides, e.g., decyl, dodecyl, lauryl, myristyl, and stearyl chlorides, bromides, and iodides; and aryl(C₁-C₄)alkyl halides, e.g., benzylchloride and phenethyl bromide. Such salts permit the preparation of both water-soluble and oil-soluble compounds of the present invention.

When the compounds of the present invention possess a free acid form, a pharmaceutically acceptable base addition salt can be prepared by reacting the free acid form of the compound with a pharmaceutically acceptable organic or inorganic base. Non-exclusive examples of base addition salts include, but are not limited to alkali metal hydroxides including potassium, sodium, and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are aluminum salts of the compounds of the present invention. Further base salts of the present invention include, but are not limited to: copper, ferric, ferrous, lithium, magnesium, manganic, manganous, potassium, sodium, and zinc salts. Organic base salts include but are not limited to, salts of primary, secondary, and tertiary amines, substituted amines including naturally occurring substituted amines, cyclic amines, e.g., ammonium, tetramethylammonium, tetraethylammonium, methylamine, dimethylamine, trimethylamine, triethylamine, and ethylamine; and basic ion exchange resins, e.g., arginine, betaine, caffeine, chloroprocaine, choline, N,N′-dibenzylethylenediamine, dicyclohexylamine, diethanolamine, 2-diethylaminoethanol, 2-dimethylaminoethanol, ethanolamine, ethylenediamine, N-ethylmorpholine, N-ethylpiperidine, glucamine, and glucosamine. See, e.g., Berge S. M., et. al., Pharmaceutical Salts, J. of Pharma. Sci., 66:1, (1977). It should be recognized that the free acid forms will typically differ from their respective salt forms somewhat in physical properties such as solubility in polar solvents, but otherwise the salts are equivalent to their respective free acid forms for the purposes of the present invention.

All of the salt forms are within the scope of the compounds useful in the method of the present invention. Conventional concentration or crystallization techniques can be employed to isolate the salts.

The compounds and salts of the present invention may inherently form solvates, including hydrated forms, with pharmaceutically acceptable solvents. A solvate refers to a molecular complex of a compound represented by Formula (1A) including pharmaceutically acceptable salts thereof, with one or more solvent molecules. In general, the solvated forms, including hydrated forms, are equivalent to unsolvated forms and are intended to be encompassed within the scope of the present invention. Solvents that are commonly used in the pharmaceutical art, which are known to be innocuous to the recipient include water, ethanol, methanol, isopropanol, dimethylysulfoxide (DMSO), ethyl acetate, acetic acid, or ethanolamine, and the like. Although pharmaceutically acceptable solvents are preferred, other solvents may be used and then displaced with a pharmaceutically acceptable solvent to acquire certain polymorphs. A hydrate refers to the complex where the solvent molecule is water. Solvates, including hydrates, are considered compositions of the compound of the present invention.

It is also possible that the intermediates and compounds of the present invention may exist in different tautomeric forms. Tautomers refer to organic compounds that are interconvertible, i.e., when a chemical reaction results in a formal migration of a proton accompanied by a switch of a single bond and adjacent double bond (e.g., enol/keto, amide/imidic acid, and amine/imine forms) or as illustrated below

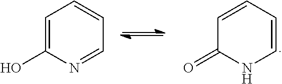

See, e.g., Katritzky, A. R., et. al., The Tautomerism of Heterocycles, Academic Press, New York, (1976). All such tautomeric forms are embraced within the scope of the invention.

The present invention also includes isotopically-labelled compounds, which are identical to those recited for the compound of Formula (1A), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively. Compounds of Formula (1A) which contain the aforementioned isotopes and/or other isotopes of other atoms are within the scope of this invention.

Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Positron emitting isotopes such as ¹⁵O, ¹³N, ¹¹C, and ¹⁸F are useful for positron emission tomography (PET) studies to examine substrate occupancy. Isotopically labeled compounds of this invention thereof can generally be prepared by carrying out the procedures disclosed herein, by substituting a readily available isotopically labelled reagent for a non-isotopically labeled reagent.

Compounds of the present invention are useful for treating diseases, conditions and/or disorders mediated by the activation of glucokinase; therefore, another embodiment of the present invention is a pharmaceutical composition comprising a therapeutically effective amount of a compound of the present invention, or a pharmaceutically acceptable salt thereof, and a pharmaceutically acceptable excipient, diluent or carrier. The compounds of the present invention (including the compositions and processes used therein) may also be used in the manufacture of a medicament for the therapeutic applications described herein.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations can be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handled product. The pharmaceutical compositions also include solvates and hydrates of the Formula (1A) compounds.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders mediated by the activation of glucokinase in a mammal that includes administering to a mammal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders that benefit from the activation of glucokinase which include: eating disorders (e.g., binge eating disorder, anorexia, bulimia, weight loss or control and obesity), prevention of obesity and insulin resistance by glucokinase expression in skeletal muscle of transgenic mice (Otaegui, P. J., et. al., The FASEB Journal, 17; 2097-2099, (2003)); and Type II diabetes, insulin resistance syndrome, insulin resistance, and hyperglycemia (Poitout, V., et. al., “An integrated view of β-cell dysfunction in type-II diabetes”, Annul. Rev. Medicine, 47; 69-83, (1996)).

One aspect of the present invention is the treatment of Type II diabetes, progression of disease in Type II diabetes, metabolic syndrome (Syndrome X or a combination of elevated blood glucose, hypertension, obesity, decreased HDL cholesterol, and elevated triglycerides), hyperglycemia, impaired glucose tolerance (a pre-diabetic state of dysglycemia associated with insulin resistance), glucosuria (abnormal condition of osmotic diuresis due to excretion of glucose by the kidneys), cataracts, diabetic neuropathy, diabetic nephropathy, diabetic retinopathy, obesity; conditions exacerbated by obesity; hypertension; dyslipidemia; hyperinsulinemia (excess circulating blood insulin often associated with metabolic syndrome and NIDDM), and diabetic macular edema. The preferred disease, disorder, or condition to be treated is Type II diabetes, hyperglycemia, and reducing blood glucose. Most preferred is Type II diabetes.

Diabetes is generally defined as a syndrome characterized by disordered metabolism and inappropriately high blood glucose (hyperglycemia) resulting from either low levels of the hormone insulin or from abnormal resistance to insulin's effects coupled with inadequate levels of insulin secretion to compensate. Diabetes is generally characterized as three main forms: (1) Type I, (2) Type II, and (3) gestational diabetes. Type I diabetes is usually due to autoimmune destruction of the pancreatic beta cells. Type II diabetes is characterized by insulin resistance in target tissues. This causes a need for abnormally high amounts of insulin and diabetes develops when the beta cells cannot meet this demand. Gestational diabetes is similar to Type II diabetes in that it involves insulin resistance; the hormones of pregnancy can cause insulin resistance in women genetically predisposed to developing this condition, and typically resolves with delivery of the child. However, Types I and II are chronic conditions. Type 1 diabetes, in which insulin is not secreted by the pancreas, is directly treatable with insulin, although dietary and other lifestyle adjustments are part of disease management. Type II diabetes may be managed with a combination of diet and pharmaceutical products (e.g., medicaments), and frequently, insulin supplementation. Diabetes can cause many complications. Acute complications include hypoglycemia, hyperglycemia, ketoacidosis or nonketotic hyperosmolar coma. Serious long-term complications include, but are not limited to: cardiovascular disease, renal failure, retinal damage, decreased blood circulation, nerve damage, and hypertension.

In yet another aspect of the present invention is the treatment of diabetes related disorders, such as metabolic syndrome. Metabolic syndrome includes diseases, conditions or disorders such as dyslipidemia, hypertension, insulin resistance, coronary artery disease, obesity, and heart failure. For more detailed information on Metabolic Syndrome, see, e.g., Zimmet, P. Z., et al., “The Metabolic Syndrome: Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Preferably, administration of the compounds of the present invention provides a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the present invention may also provide a statistically significant (p<0.05) reduction in glucose serum levels.

For a normal adult human having a body weight of about 100 kg, a dosage in the range of from about 0.001 mg to about 10 mg per kilogram body weight is typically sufficient, preferably from about 0.01 mg/kg to about 5.0 mg/kg, more preferably from about 0.01 mg/kg to about 1 mg/kg. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The compounds of this invention may also be used in conjunction with additional pharmaceutical agents for the treatment of the diseases, conditions and/or disorders described herein. Therefore, methods of treatment that include administering compounds of the present invention in combination with additional pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-obesity agents include cannabinoid-1 (CB-1) antagonists (such as rimonabant), 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆ (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include CB-1 antagonists (e.g., rimonabant, taranabant, surinabant, otenabant, SLV319 (CAS No. 464213-10-3) and AVE1625 (CAS No. 358970-97-5)), gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

Suitable anti-diabetic agents include an acetyl-CoA carboxylase-2 (ACC-2) inhibitor, a phosphodiesterase (PDE)-10 inhibitor, a diacylglycerol acyltransferase (DGAT) 1 or 2 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Preferred anti-diabetic agents are metformin and DPP-IV inhibitors (e.g., sitagliptin, vildagliptin, alogliptin and saxagliptin).

Suitable antihyperglycemic agents include, but are not limited to, alpha-glucosidase inhibitors (i.e., acarbose), biguanides, insulin, insulin secretagogues (i.e., sulfonureas (i.e., gliclazide, glimepiride, glyburide) and nonsulfonylureas (i.e., nateglinide and repaglinide)), thiazolidinediones (i.e. pioglitazone, rosiglitazone), and the like.

Suitable lipid lowering agents include, but are not limited to, HMGCoA reductase inhibitors, fibrates, microsomal triglyceride transfer protein inhibitors, cholesterol transfer protein inhibitors, acyl transfer protein inhibitors, low density lipid antioxidants, and the like.

Suitable antihypertensive agents include, but are not limited to, diuretics, adrenergic beta-antagonists, adrenergic alpha-antagonists, angiotensin-converting enzyme inhibitors, calcium channel blockers, ganglionic blockers, vasodilators, and the like.

All of the above recited U.S. patents and publications are incorporated herein by reference.

According to the methods of the invention, when a compound of the present invention and at least one other pharmaceutical agent are administered together, such administration can be sequential in time or simultaneous with the simultaneous method being generally preferred. For sequential administration, a compound of the present invention and the additional pharmaceutical agent can be administered in any order. It is generally preferred that such administration be oral. It is especially preferred that such administration be oral and simultaneous. When a compound of the present invention and the additional pharmaceutical agent are administered sequentially, the administration of each can be by the same or by different methods, for example, tablet and syrup or capsule and parenteral injection or infusion. Administration and dosing will be determined by the prescribing practitioner.

The starting materials and reagents used in preparing these compounds are either available from commercial suppliers such as the Aldrich Chemical Company (Milwaukee, Wis.), Bachem (Torrance, Calif.), Sigma (St. Louis, Mo.), Acros Organics (Geel, Belgium), or Lancaster Synthesis Ltd. (Morecambe, United Kingdom) or may be prepared by methods well known to a person of ordinary skill in the art, following procedures described in such standard references as Fieser and Fieser's Reagents for Organic Synthesis, Vols. 1-17, John Wiley and Sons, New York, N.Y., (1991); Rodd's Chemistry of Carbon compounds, Vols. 1-5 and supps., Elsevier Science Publishers, (1989); Organic Reactions, Vols. 1-40, John Wiley and Sons, New York, N.Y., (1991); March J., Advanced Organic Chemistry, 4th ed., John Wiley and Sons, New York, N.Y.; and Larock, Comprehensive Organic Transformations, VCH Publishers, New York, (1989). Anhydrous tetrahydrofuran (THF), methylene chloride (CH₂Cl₂), and N,N-dimethylformamide (DMF) may be purchased from Aldrich in Sure-Seal bottles and used as received. Solvents may be purified using standard methods known to those skilled in the art, unless otherwise indicated. Further, starting materials were obtained from commercial suppliers and used without further purification, unless otherwise indicated.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed using glass-backed silica gel 60 F 254 precoated plates (Merck Art 5719) and eluted with appropriate solvent ratios (v/v). Reactions were assayed by TLC or LCMS and terminated as judged by the consumption of starting material. Visualization of the TLC plates was done with UV light (254 nM wavelength) or with an appropriate TLC visualizing solvent and activated with heat. Flash column chromatography (Still et al., J. Org. Chem. 43, 2923, (1978)) was performed using silica gel 60 (Merck Art 9385) or various MPLC systems, such as Biotage or ISCO purification system.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography (TLC)), recrystallization, and differential (i.e., liquid-liquid) extraction techniques. Biotage materials were purchased from Biotage AB (Charlottesville, Va.).

The compound structures in the examples below were confirmed by one or more of the following methods: proton magnetic resonance spectroscopy, mass spectroscopy, and elemental microanalysis. Proton magnetic resonance (¹H NMR) spectra were determined using a Bruker or Varian spectrometer operating at a field strength of 300 or 400 megahertz (MHz). Chemical shifts are reported in parts per million (PPM, δ) downfield from an internal tetramethylsilane standard. Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Mass spectra (MS) data were obtained using Agilent mass spectrometer or Waters Micromass spectrometer with atmospheric pressure chemical or electron spray ionization. Method: Acquity HPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%) Elemental microanalyses were performed by Atlantic Microlab Inc. and gave results for the elements stated within ±0.4% of the theoretical values.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

The following examples provide a more detailed description of the process conditions. It is to be understood, however, that the invention, as fully described herein and as recited in the claims, is not intended to be limited by the details of the following schemes or modes of preparation.

### Example 1

### (S)-3-cyclopentyl-N-(5-methylpyrazin-2-yl)-2-(2-oxo-4-(trifluoromethyl)pyridin-1(2H)-yl)propanamide (1)

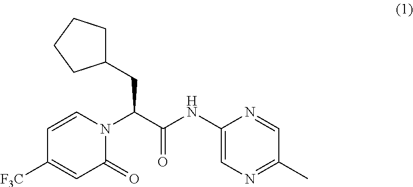

### Intermediate: (R)-3-cyclopentyl-2-hydroxypropanoic acid (1a)

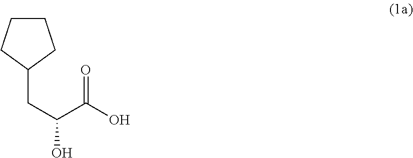

To a stirred solution of (R)-2-amino-3-cyclopentylpropanoic acid (5.00 g) and 1 M H₂SO₄ (45.1 mL) at 0° C., was added a solution of NaNO₂ (3.12 g) in H₂O (15.6 mL) drop wise over 10 minutes. The reaction mixture was stirred for 3 hours at 0° C., then for 2 hours at room temperature. The solution was then extracted with ether (3 times). The combined organic extracts were dried over MgSO₄, filtered and the filtrate was concentrated to give (R)-3-cyclopentyl-2-hydroxypropanoic acid (1a) (2.36 g). ¹H NMR (400 MHz, CDCl₃) δ 4.26-4.28 (1H), 1.99-2.07 (1H), 1.76-1.81 (4H), 1.60-1.62 (4H), 1.12-1.16 (2H); LCMS for C₈H₁₄O₃ m/z 157.1 (M−H)⁻.

### Intermediate: (R)-methyl 3-cyclopentyl-2-hydroxypropanoate (1b)

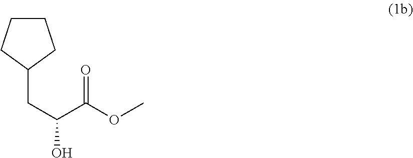

To a stirred solution of (R)-3-cyclopentyl-2-hydroxypropanoic acid (1a) (2.36 g) in anhydrous methanol (15 mL) at room temperature was added SOCl₂ (1.64 mL). The resulting mixture was heated at reflux for 2 hours. It was then cooled and concentrated under reduced pressure. The residue was partitioned between ethyl acetate and aqueous saturated NaHCO₃ solution. The biphasic mixture was separated and the aqueous portion was extracted with ethyl acetate. The combined extracts were dried over MgSO₄, filtered and the filtrate was concentrated under reduced pressure. The resulting residue was purified by flash column chromatography (silica gel, heptanes/ethyl acetate) to provide (R)-methyl 3-cyclopentyl-2-hydroxypropanoate (1b) as clear oil (1.5 g). ¹H NMR (400 MHz, CDCl₃) δ 4.15-4.20 (1H), 3.77 (3H), 2.62-2.63 (1H), 1.97-2.05 (1H), 1.49-1.86 (8H), 1.06-1.17 (2H); LCMS for C₉H₁₆O₃ m/z 171.6 (M)⁺.

### Preparation of Intermediate: (R)-methyl 3-cyclopentyl-2-(trifluoromethylsulfonyloxy)-propanoate (1c)

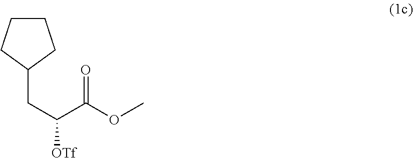

To a stirred solution of (R)-methyl 3-cyclopentyl-2-hydroxypropanoate (1b) (0.050 g) in anhydrous CH₂Cl₂ (3 mL) at 0° C. under nitrogen was added 2,6-lutidine (0.064 mL) followed by drop wise trifluoromethanesulfonic anhydride (0.083 mL). After stirring for 45 minutes at the same temperature, methyl tert-butyl ether was added and the mixture was thoroughly (3 times) washed with a mixture of brine and aqueous 1N HCl (3:1). The organic extracts were dried over MgSO₄, filtered, and the filtrate concentrated to give (R)-methyl 3-cyclopentyl-2-(trifluoromethylsulfonyloxy)propanoate (1c) as a tan oil. ¹H NMR (400 MHz, CDCl₃) δ 5.09-5.12 (1H), 3.81 (3H), 1.97-2.09 (1 H), 1.70-1.96 (4H), 1.47-1.66 (4H), 1.03-1.19 (2H).

### Intermediate: (S)-methyl 3-cyclopentyl-2-(2-oxo-4-(trifluoromethyl)pyridin-1(2H)-yl)propanoate (1d)

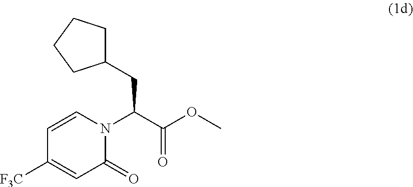

To a stirred solution of 4-(trifluoromethyl)pyridin-2(1H)-one (946 mg, 5.8 mmol) in anhydrous THF (40 mL) at room temperature under nitrogen, was added a solution of lithium bis(trimethylsilyl)amide (5.2 mL, 5.2 mmol, 1.0M in THF). After stirring for 40 minutes, a solution of (R)-methyl 3-cyclopentyl-2-(trifluoromethylsulfonyloxy)propanoate (1c) (1.77 g, 5.81 mmol) in anhydrous THF (10 mL) was added. The reaction was then stirred for 1.5 hours and quenched with aqueous saturated ammonium chloride and diluted with brine and ethyl acetate. The aqueous layer was extracted with ethyl acetate, and the combined organics were dried over sodium sulfate, filtered, and evaporated. The residue was purified (Combi-flash, Redi-sep 80 g, 25% ethyl acetate/heptane gradient to 80% ethyl acetate/heptane, 254 nm detection, 240 nm monitoring, all fractions collected, the product has weak uv). The product fractions were combined, evaporated, and dried under reduced pressure to provide (S)-methyl 3-cyclopentyl-2-(2-oxo-4-(trifluoromethyl)pyridin-1(2H)-yl)propanoate (1d) (1.33 g). ¹H NMR (400 MHz, CDCl₃) δ 7.46-7.45 (1H), 6.81 (1H), 6.33-6.31 (1H), 5.60-5.56 (1H), 3.72 (3H), 2.12-2.09 (1H), 2.03-1.99 (1H), 1.79-1.46 (7H), 1.23-1.05 (2H); LCMS for C₁₅H₁₈F₃NO₃ m/z 318.1 (M+H)⁺.

### Final Preparation: (S)-3-cyclopentyl-N-(5-methylpyrazin-2-yl)-2-(2-oxo-4-(trifluoromethyl)pyridin-1(2H)-yl)propanamide (1)

To a stirred solution of 2-amino-5-methylpyrazine (186 mg, 1.70 mmol) in a 4-dram vial in dry toluene, was added AlMe₃ (0.85 mL, 2 M in toluene). After stirring for 45 minutes at room temperature, a solution of (S)-methyl 3-cyclopentyl-2-(2-oxo-4-(trifluoromethyl)pyridin-1(2H)-yl)propanoate (1d) (250 mg, 0.788 mmol) in 1,2-dichloroethane was added, and the reaction was sealed and heated at 80° C. The reaction was cooled, and the residue was diluted with dichloromethane and 0.5 M Rochelle salt, shaken, and allowed to stir for 60 minutes. The mixture was filtered through an Autovial filter to remove insoluble material. Brine was added, and the organic layer was separated and evaporated. The residue was purified (Combi-flash, Redi-sep 40 g, 20% ethyl acetate/heptane gradient to 1:1 ethyl acetate/heptane). The product fractions were combined, evaporated, and dried under high vacuum. The resulting solid was triturated and stirred with heptane and ether, the resulting solid was collected by filtration and dried under reduced pressure to provide (S)-3-cyclopentyl-N-(5-methylpyrazin-2-yl)-2-(2-oxo-4-(trifluoromethyl)pyridin-1(2H)-yl)propanamide (1) (0.148 g). ¹H NMR (400 MHz, CDCl₃) δ 10.5 (1H), 9.33 (1H), 8.09 (1H), 7.98 (1H), 7.37 (1H), 6.46 (1H), 6.23 (1H), 2.50 (3H), 2.28 (1H), 1.99 (1H), 1.83 (2H), 1.62 (2H), 1.49 (2H), 1.22 (3H); LCMS for C₁₉H₂₁F₃N₄O₂ m/z 395.30 (M+H)⁺.

### Example 2

### (S)-3-cyclopentyl-N-(5-methylpyridin-2-yl)-2-(2-oxo-3-(trifluoromethyl)pyridin-1(2H)-yl)propanamide (2)

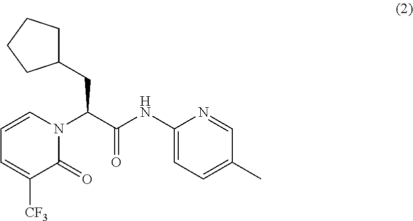

To a stirred solution of 2-amino-5-picoline (82.2 mg, 0.760 mmol) in 1,2-dichloroethane (3 mL) at 0° C. was added Al(CH₃)₂Cl (1.0 M in hexanes, 0.760 mL, 0.760 mmol). The mixture was stirred at room temperature for 15 minutes, and then the solution of (S)-methyl 3-cyclopentyl-2-(2-oxo-3-(trifluoromethyl)pyridin-1(2H)-yl)propanoate (30 mg, 0.095 mmol) in 0.5 mL of 1,2-dichloroethane was added. The (S)-methyl 3-cyclopentyl-2-(2-oxo-3-(trifluoromethyl)pyridin-1(2H)-yl)propanoate was made in a similar manner to that of (1d) but starting with 3-(trifluoromethyl)pyridin-2(1H)-one. The reaction mixture was stirred at room temperature overnight. The reaction mixture was slowly quenched with 20% aqueous potassium sodium tartrate tetrahydrate (5 mL), diluted with water (30 mL) and extracted with CHCl₃ (30 mL). The combined organic extracts were dried (MgSO₄) and concentrated. The resulting residue was purified on a Biotage column (12+S), eluting with heptane/ethyl acetate 0-30% (3 CV), 30% (5CV), 30-70% (1 CV), 70% (4 CV), to provide (S)-3-cyclopentyl-N-(5-methylpyridin-2-yl)-2-(2-oxo-3-(trifluoromethyl)pyridin-1(2H)-yl)propanamide (2) as a white solid (26 mg). ¹H NMR (400 MHz, CDCl₃) δ 9.18 (1H), 8.10 (1H), 7.84 (1H), 7.82 (1H), 7.74 (1H), 7.43 (1H), 6.34 (1H), 5.81 (1H), 2.25 (3H), 2.24 (1H), 1.94 (1 H), 1.71-1.81 (3H), 1.61 (2H), 1.49 (2H), 1.15-1.525 (2H); LCMS for C₂₀H₂₂F₃N₃O₂ m/z 394.2 (M+H)⁺.

Examples 3-24, 27, and 29-37 were made in an analogous manner to that of Examples 1 and 2 using appropriate starting materials.

### Example 3

### (S)-3-cyclopentyl-N-(5-methylpyridin-2-yl)-2-(2-oxo-4-(trifluoromethyl)pyridin-1(2H)-yl)propanamide (3)

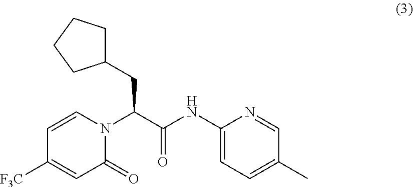

¹H NMR (400 MHz, CDCl₃) δ 9.49 (1H), 8.12 (1H), 7.98-8.00 (1H), 7.79-7.81 (1H), 7.47-7.49 (1H), 7.14 (1H), 6.38-6.40 (1H), 5.88-5.91 (1H), 2.27 (3H), 2.21-2.27 (1 H), 1.92-1.99 (1H), 1.68-1.84 (3H), 1.46-1.63 (4H), 1.12-1.22 (2H); LCMS for C₂₀H₂₂N₃O₂F₃ m/z 394.5 (M+H)⁺.

### Example 4

### (S)-3-cyclopentyl-N-(1-methyl-1H-pyrazol-3-yl)-2-(2-oxo-4-(trifluoromethyl)pyridin-1(2H)-yl)propanamide (4)

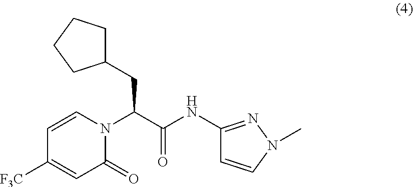

¹H NMR (400 MHz, CDCl₃) δ 10.78 (1H), 8.03 (1H), 7.55 (1H), 7.22 (1H), 6.63 (1H), 6.40 (1H), 6.15 (1H), 3.76 (3H), 2.23 (1H), 1.86 (4H), 151 (2H), 1.48 (2H), 1.22 (2H); LCMS for C₁₈H₂₁F₃N₄O₂ m/z 383.30 (M+H).

### Example 5

### (S)-3-cyclopentyl-N-(5-methylpyrazin-2-yl)-2-(2-oxo-3-(trifluoromethyl)pyridin-1(2H)-yl)propanamide (5)

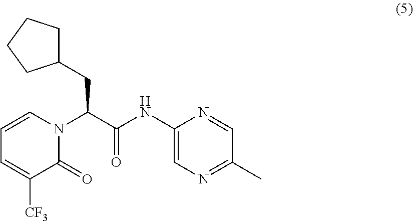

¹H NMR (400 MHz, CDCl₃) δ 9.45 (1H), 7.88 (1H), 7.84 (1H), 7.79 (1H), 6.40 (1H), 6.01 (1H), 2.41 (3H), 2.28-2.33 (1H), 1.98-1-90 (2H), 1.88 (2H), 1.71-1.62 (1H), 1.60-1.51 (2H), 1.30-1.18 (4H); LCMS for C₁₉H₂₁F₃N₄O₂ m/z 395.3 (M+H)⁺.

### Example 6

### (S)-3-cyclopentyl-N-(5-methylpyridin-2-yl)-2-(2-oxo-5-(trifluoromethyl)pyridin-1(2H)-yl)propanamide (6)

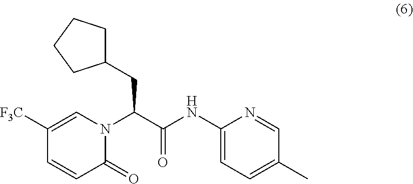

¹H NMR (400 MHz, CDCl₃) δ 9.45 (1H), 9.05 (1H), 7.89 (2H), 7.79 (1H), 6.41 (1H), 6.01 (1H), 2.41 (3H), 2.30 (1H), 1.93 (1H), 1.80 (2H), 1.62 (2H), 1.50 (2H), 1.25 (2H), 1.23 (2H); LCMS for C₂₀H₂₂F₃N₃O₂ m/z 394.30 (M+H)⁺.

### Example 7

### (S)-3-cyclopentyl-N-(5-methylpyrazin-2-yl)-2-(2-oxo-5-(trifluoromethyl)pyridin-1(2H)-yl)propanamide (7)

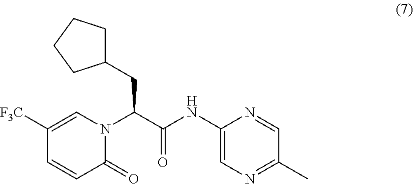

¹H NMR (400 MHz, CDCl₃) δ 9.75 (1H), 9.29 (1H), 8.10 (2H), 7.50 (1H), 6.84 (1H), 5.96 (1H), 2.50 (3H), 2.29 (1H), 1.97 (1H), 1.79 (2H), 1.61 (2H), 1.50 (2H), 1.23 (3 H); LCMS for C₁₉H₂₁F₃N₄O₂ m/z 395.30 (M+H)⁺.

### Example 8

### (S)-3-cyclopentyl-2-(2-oxo-5-(trifluoromethyl)pyridin-1(2H)-yl)-N-(pyrazin-2-yl)propanamide (8)

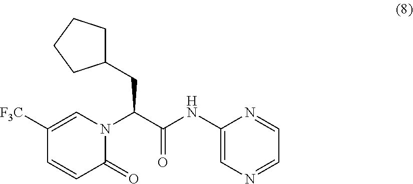

¹H NMR (400 MHz, CDCl₃) δ 9.49 (1H), 9.43 (1H), 8.34 (1H), 8.25 (1H), 7.98 (1H), 7.49-7.51 (1H), 6.77-6.79 (1H), 5.79-5.86 (1H), 2.30-2.36 (1H), 1.93-1.99 (1H), 1.71-1.81 (2H), 1.51-1.62 (4H), 1.19-1.25 (2H); LCMS for C₁₈H₁₉N₄O₂F₃ m/z 381.1 (M+H)⁺.

### Example 9

### (S)-3-cyclopentyl-2-(2-oxo-5-(trifluoromethyl)pyridin-1(2H)-yl)-N-(pyrimidin-4-yl)propanamide (9)

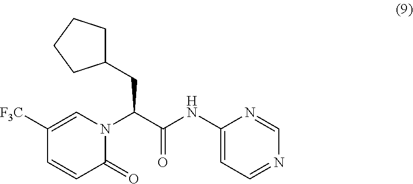

¹H NMR (400 MHz, CDCl₃) δ 9.64 (1H), 8.87 (1H), 8.61-8.62 (1H), 8.06-8.07 (1H), 7.95 (1H), 7.50-7.52 (1H), 6.81-6.83 (1H), 5.82-5.86 (1H), 2.26-2.34 (1H), 1.92-1.99 (1H), 1.61-1.81 (6H), 1.51-1.56 (1H), 1.19-1.24 (2H); LCMS for C₁₈H₁₉N₄O₂F₃ m/z 381.1 (M+H)⁺.

### Example 10

### (S)-3-cyclopentyl-N-(1-ethyl-1H-pyrazol-3-yl)-2-(2-oxo-5-(trifluoromethyl)pyridin-1(2H)-yl)propanamide (10)

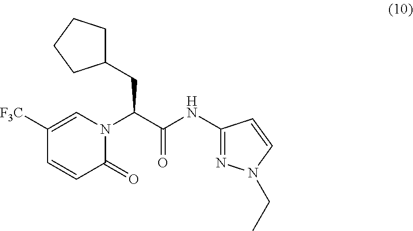

¹H NMR (400 MHz, CDCl₃) δ 9.60 (1H), 8.11 (1H), 7.44-7.46 (1H), 7.26-7.27 (1H), 6.86-6.88 (1H), 6.60 (1H), 5.85-5.88 (1H), 4.02-4.07 (2H), 2.25-2.32 (1H), 1.72-1.92 (3H), 1.55-1.65 (2H), 1.41-1.51 (6H), 1.13-1.21 (2H); LCMS for C₁₉H₂₃N₄O₂F₃ m/z 397.2 (M+H)⁺.

### Example 11

### (S)—N-(1-benzyl-1H-pyrazol-3-yl)-3-cyclopentyl-2-(2-oxo-5-(trifluoromethyl)pyridin-1(2H)-yl)propanamide (11)

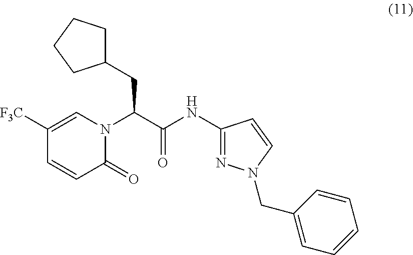

¹H NMR (400 MHz, CDCl₃) δ 9.80 (1H), 8.11 (1H), 7.27-2.35 (5H), 7.15-7.21 (2H), 6.79-6.82 (1H), 6.67 (1H), 5.87-5.90 (1H), 5.13-5.22 (2H), 2.20-2.27 (1H), 1.68-1.90 (4H), 1.43-1.60 (4H), 1.10-1.19 (2H); LCMS for C₂₄H₂₅N₄O₂F₃ m/z 459.2 (M+H)⁺.

### Example 12

### (S)-3-cyclopentyl-2-(2-oxo-5-(trifluoromethyl)pyridin-1(2H)-yl)-N-(pyrimidin-2-yl)propanamide (12)

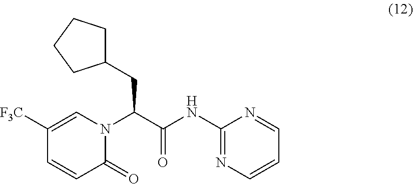

¹H NMR (400 MHz, CDCl₃) δ 9.43 (1H), 8.61-8.63 (2H), 8.01 (1H), 7.45-7.48 (1H), 7.03-7.06 (1H), 6.71-6.74 (1H), 6.06 (1H) 2.30-2.36 (1H), 1.92-1.99 (1H), 1.45-1.84 (7H), 1.11-1.28 (2H); LCMS for C₁₈H₁₉N₄O₂F₃ m/z 381.0 (M+H)⁺.

### Example 13

### (S)-3-cyclopentyl-2-(2-oxo-4-(trifluoromethyl)pyridin-1(2H)-yl)-N-(pyrazin-2-yl)propanamide (13)

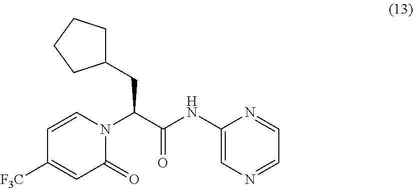

¹H NMR (400 MHz, CDCl₃) δ 10.22 (1H), 9.45 (1H), 8.34 (1H), 8.25 (1H), 7.87-7.89 (1 H), 7.25-7.26 (1H), 6.45-6.47 (1H), 6.08-6.12 (1H), 2.25-2.32 (1H), 1.96-2.04 (1H), 1.72-1.84 (3H), 1.46-1.64 (4H), 1.14-1.26 (2H); LCMS for C₁₈H₁₉N₄O₂F₃ m/z 381.4 (M+H)⁺.

### Example 14

### (S)-3-cyclopentyl-2-(2-oxo-4-(trifluoromethyl)pyridin-1(2H)-yl)-N-(pyrimidin-4-yl)propanamide (14)

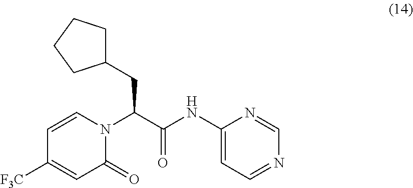

¹H NMR (400 MHz, CDCl₃) major rotamer δ 10.29 (1H), 8.86 (1H), 8.60-8.62 (1H), 8.06-8.08 (1H), 7.81-7.83 (1H), 7.29 (1H), 6.45-6.47 (1H), 6.04-6.08 (1H), 2.22-2.32 (1H), 1.96-2.03 (1H), 1.68-1.82 (3H), 1.46-1.62 (4H), 1.12-1.24 (2H); LCMS for C₁₈H₁₉N₄O₂F₃ m/z 381.4 (M+H)⁺.

### Example 15

### (S)-3-cyclopentyl-N-(1-ethyl-1H-pyrazol-3-yl)-2-(2-oxo-4-(trifluoromethyl)pyridin-1(2H)-yl)propanamide (15)

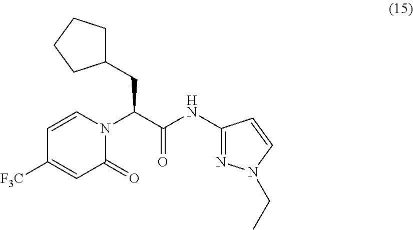

¹H NMR (400 MHz, CDCl₃) δ 10.43 (1H), 7.98-8.00 (1H), 7.41 (1H), 7.26 (1H), 6.62 (1 H), 6.38-6.40 (1H), 6.07-6.11 (1H), 4.01-4.07 (2H), 2.22-2.29 (1H), 1.72-1.94 (4H), 1.46-1.63 (4H), 1.39-1.43 (3H), 1.13-1.24 (2H); LCMS for C₁₉H₂₃N₄O₂F₃ m/z 397.5 (M+H)⁺.

### Example 16

### (S)-3-cyclopentyl-N-(1-methyl-1H-pyrazol-3-yl)-2-(2-oxo-5-(trifluoromethyl)pyridin-1(2H)-yl)propanamide (16)

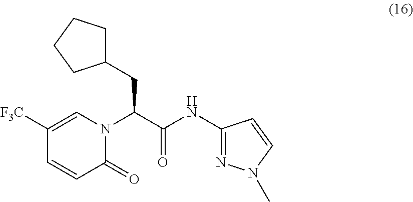

LCMS for C₁₈H₂₁F₃N₄O₂ m/z 383 (M+H)⁺; HPLC t_{R}=0.51 min (100%), Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 17

### (S)-3-cyclopentyl-2-(2-oxo-5-(trifluoromethyl)pyridin-1(2H)-yl)-N-(5-(trifluoromethyl)pyridin-2-yl)propanamide (17)

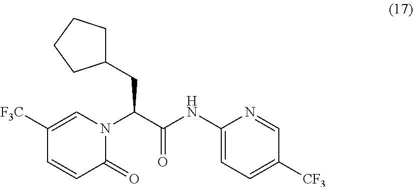

LCMS for C₂₀H₁₉F₆N₃O₂ m/z 448 (M+H)⁺; HPLC t_{R}=0.62 min (100%). Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 18

### (S)-3-cyclopentyl-N-(isoxazol-3-yl)-2-(2-oxo-5-(trifluoromethyl)pyridin-1(2H)-yl)propanamide (18)

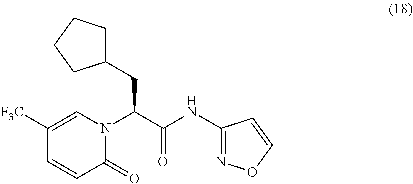

LCMS for C₁₇H₁₈F₃N₃O₃ m/z 370 (M+H)⁺; HPLC t_{R}=0.52 min (100%). Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 19

### (S)-3-cyclopentyl-2-(2-oxo-5-(trifluoromethyl)pyridin-1(2H)-yl)-N-(quinolin-2-yl)propanamide (19)

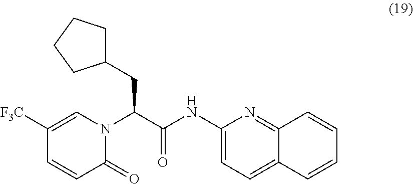

LCMS for C₂₃H₂₂F₃N₃O₂ m/z 430 (M+H)⁺; HPLC t_{R}=0.56 min (100%). Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 20

### (S)-3-cyclopentyl-N-(5-methoxypyrazin-2-yl)-2-(2-oxo-5-(trifluoromethyl)pyridin-1(2H)-yl)propanamide (20)

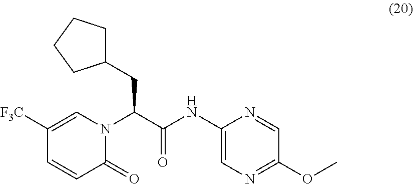

LCMS for C₁₉H₂₁F₃N₄O₃ m/z 411 (M+H)⁺; HPLC t_{R}=0.56 min (100%). Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 21

### (S)-3-cyclopentyl-2-(2-oxo-5-(trifluoromethyl)pyridin-1(2H)-yl)-N-(pyridin-2-yl)propanamide (21)

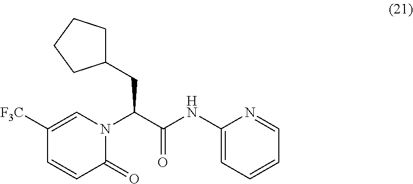

LCMS for C₁₉H₂₀F₃N₃O₂ m/z 380 (M+H)⁺; HPLC t_{R}=0.50 min (100%). Method: Acquity UPLC with chromatography performed on a Waters BEH C18 column (2.1×30 mm, 1.75 μm) at 60° C. The mobile phase was a binary gradient of acetonitrile (containing 0.05% trifluoroacetic acid) and water (5-95%).

### Example 22

### (S)-3-cyclopentyl-N-(5-methylpyrazin-2-yl)-2-(6-oxo-4-(trifluoromethyl)pyrimidin-1(6H)-yl)propanamide (22)

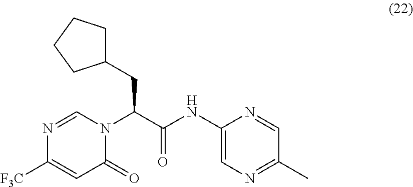

¹H NMR (400 MHz, CDCl₃) δ 9.85-9.87 (1H) 9.31 (1H) 8.71 (1H) 8.08 (1H) 7.25 (1H) 5.98-6.01 (1H) 2.52 (3H) 2.27-2.34 (1H) 2.03-2.11 (1H) 1.45-1.85 (7H) 1.14-1.26 (2 H); LCMS for C₁₈H₂₀N₅O₂F₃ m/z 396.1 (M+H)⁺.

### Example 23

### (S)-3-cyclopentyl-N-(5-methylpyridin-2-yl)-2-(6-oxo-4-(trifluoromethyl)pyrimidin-1(6H)-yl)propanamide (23)

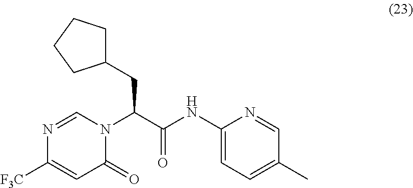

¹H NMR (400 MHz, CDCl₃) δ 10.05 (1H), 8.66 (1H), 8.12 (1H), 8.02-8.04 (1H), 7.51-7.54 (1H), 7.18 (1H), 5.86-5.90 (1H), 2.29 (3H), 2.20-2.27 (1H), 2.00-2.07 (1H), 1.69-1.80 (3H), 1.45-1.60 (4H), 1.11-1.20 (2H); LCMS for C₁₉H₂₁N₄O₂F₃ m/z 395.09 (M+H)⁺.

### Example 24

### (S)-3-cyclopentyl-N-(1-methyl-1H-pyrazol-3-yl)-2-(6-oxo-4-(trifluoromethyl)pyrimidin-1(6H)-yl)propanamide (24)

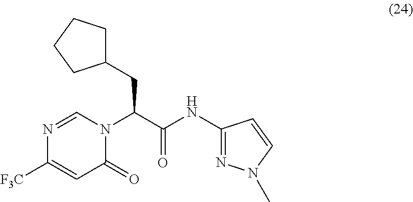

¹H NMR (400 MHz, CDCl₃) δ 10.08 (1H), 8.76 (1H), 7.43 (1H), 7.23-7.25 (1H), 6.61 (1H), 5.90-5.94 (1H), 3.78 (3H), 2.23-2.31 (1H), 1.94-2.01 (1H), 1.72-1.86 (3H), 1.45-1.61 (4H), 1.13-1.23 (2H); LCMS for C₁₇H₂₀N₅O₂F₃ m/z 384.14 (M+H)⁺.

### Example 25

### (S)-ethyl 2-(2-(3-cyclopentyl-2-(2-oxo-5-(trifluoromethyl)pyridin-1(2H)-yl)propanamido)thiazol-5-yl)acetate (25)

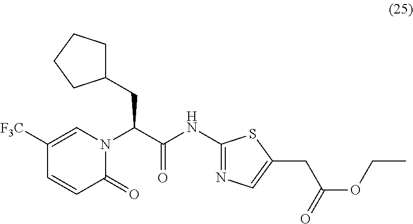

Intermediate: (S)-methyl 3-cyclopentyl-2-(2-oxo-5-(trifluoromethyl)pyridin-1(2H)-yl)propanoate (25a) was prepared as described in Scheme 2.

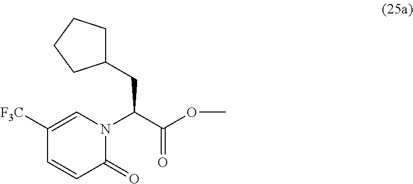

To a stirred solution of 5-(trifluoromethyl)-2-(1H)-pyridone (2.37 g) in 28 mL anhydrous THF under N₂ was added lithium bis(trimethylsilyl)amide (13.1 mL, 1M in THF). After stirring for 35 minutes at room temperature, a solution of (R)-methyl 3-cyclopentyl-2-(trifluoromethylsulfonyloxy)-propanoate (4.417 g) in 14 mL anhydrous THF was added dropwise. After stirring for 2 hours, the reaction was quenched with saturated NH₄Cl followed by brine and extracted with ethylacetate. The combined organics were dried over MgSO₄ and purified by flash chromatography (120 g, 0-60% ethylacetate in heptane) to give 3.7328 g of (S)-methyl 3-cyclopentyl-2-(2-oxo-5-(trifluoromethyl)pyridin-1(2H)-yl)propanoate (25a) as a clear oil. ¹H NMR (400 MHz, CDCl₃) δ 7.73 (1H), 7.42-7.45 (1H), 6.61-6.64 (1H), 5.58-5.62 (1H), 3.75 (3H), 2.12-2.19 (1H), 1.94-2.01 (1H), 1.47-1.82 (7H), 1.06-1.20 (2H); LCMS for C₁₅H₁₈F₃NO₃ m/z 318.1 (M+H)⁺.

### Intermediate: (S)-3-cyclopentyl-2-(2-oxo-5-(trifluoromethyl)pyridin-1(2H)-yl)propanoic acid (25b)

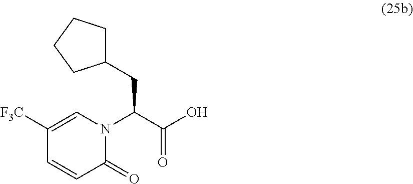

A solution of 6N HCl (12 mL) was added to (S)-methyl 3-cyclopentyl-2-(2-oxo-5-(trifluoromethyl)pyridin-1(2H)-yl)propanoate (25a) (686 mg, 2.16 mmol) and heated at 95° C. overnight thereby producing a white solid. The reaction was cooled to room temperature and diluted with ethyl acetate (20 mL) and water (10 mL). The organic layer was washed with water and brine, dried over sodium sulfate, filtered, concentrated, and dried under high vacuum to give (S)-3-cyclopentyl-2-(2-oxo-5-(trifluoromethyl)pyridin-1(2H)-yl)propanoic acid (25b) (608 mg, 2.0 mmol). ¹H NMR (400 MHz, CDCl₃) δ 7.71 (1H), 7.49-7.51 (1H), 6.70-6.72 (1H), 5.94 (2H), 5.48-5.54 (1H), 2.19-2.26 (1H), 1.99-2.09 (1H), 1.44-1.83 (7H), 1.04-1.26 (2H).

### Final preparation: (S)-ethyl 2-(2-(3-cyclopentyl-2-(2-oxo-5-(trifluoromethyl)pyridin-1(2H)-yl)propanamido)thiazol-5-yl)acetate (25)

To a stirred solution of (S)-3-cyclopentyl-2-(2-oxo-5-(trifluoromethyl)pyridin-1(2H)-yl)propanoic acid (25b) (304 mg, 1.0 mmol) in 6 mL anhydrous dichloromethane at room temperature under nitrogen was added oxalyl chloride (255 mg, 2.01 mmol) and followed by a drop of N,N-dimethylformamide. After stirring for 90 minutes, the reaction mixture was concentrated under reduced pressure and two successive portions of 1,2-dichloroethane (5 mL) were added and concentrated to give the desired acid chloride. To a solution of ethyl 2-(2-aminothiazol-5-yl)acetate (210 mg, 1.13 mmol) in dichloromethane was added a solution of the acid chloride in dichloromethane (4 mL) followed by pyridine (176 mg, 2.22 mmol). The reaction was stirred under nitrogen overnight. The reaction was diluted with ethyl acetate (20 mL) and water (20 mL), and 1M potassium dihydrogen phosphate (10 mL) was added. The layers were separated, and the organic layer was washed with brine, dried over sodium sulfate, filtered, and evaporated. The residue was purified by flash column chromatography. The fractions were combined and dried under high vacuum to give (S)-ethyl 2-(2-(3-cyclopentyl-2-(2-oxo-5-(trifluoromethyl)pyridin-1(2H)-yl)propanamido)thiazol-5-yl)acetate (25) (140 mg, 0.29 mmol). ¹H NMR (400 MHz, CDCl₃) δ 11.71 (1H), 8.16 (1H), 7.53-7.56 (1H), 7.21-7.25 (1H), 6.82 (1H), 6.14-6.18 (1H), 4.12-4.17 (2H), 3.68 (2H), 2.26-2.33 (1H), 1.90-1.97 (1H), 1.70-1.85 (3H), 1.44-1.66 (4H), 1.22-1.25 (3H), 1.13-1.20 (2H); LCMS for C₂₁H₂₄N₃O₄F₃S m/z 472.5 (M+H)⁺.

### Example 26

(S)-2-(2-(3-cyclopentyl-2-(2-oxo-5-(trifluoromethyl)pyridin-1(2H)-yl)propanamido)thiazol-5-yl)acetic acid mono acetic acid salt (26). Example 26 was prepared from Example 25 as described above, wherein the ester moiety of Example 25 was hydrolyzed to the corresponding acid.

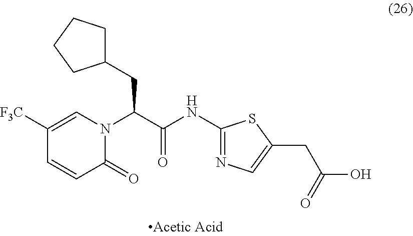

To a stirred solution of (S)-ethyl 2-(2-(3-cyclopentyl-2-(2-oxo-5-(trifluoromethyl)pyridin-1(2H)-yl)propanamido)thiazol-5-yl)acetate (25) (108 mg, 0.22 mmol) in THF, MeOH and water (1:1:1, 3 mL) at room temperature was added lithium hydroxide monohydrate (32 mg, 0.75 mmol). The resulting mixture was stirred for 40 minutes and then the solvent was removed under reduced pressure. The residue was treated with water and dichloromethane (5 mL), then ethyl acetate (20 mL). The biphasic mixture was acidified with 1N HCl. The layers were separated, and the organic layer washed with brine, dried over sodium sulfate, filtered, and evaporated under reduced pressure. The residue was purified by preparative TLC (ethyl acetate containing about 0.4% acetic acid). The product band was scraped off, crushed, and stirred for 1 hour in a 1:1 ethyl acetate/methanol (50 mL) solution. The mixture was filtered, and the residue evaporated to a cream-colored glass, which was dried under high vacuum to give (S)-2-(2-(3-cyclopentyl-2-(2-oxo-5-(trifluoromethyl)pyridin-1(2H)-yl)propanamido)thiazol-5-yl)acetic acid (26) mono acetic acid salt (61 mg). ¹H NMR (400 MHz, CD₃OD) δ 8.24 (1H), 7.67-7.69 (1H), 6.84 (1H), 6.64-6.66 (1H), 5.84-5.87 (1H), 3.57 (2H), 2.19-2.23 (2H), 1.93 (3H), 1.51-1.77 (7H), 1.15-1.25 (2H); LCMS for C₁₉H₂₀N₃O₄F₃S m/z 442.4 (M−H)⁺.

### Example 27

(S)-methyl 6-(3-cyclopentyl-2-(2-oxo-5-(trifluoromethyl)pyridin-1(2H)-yl)propanamido)nicotinate (27). Example 27 was made in an analogous manner to that of Examples 1 and 2.

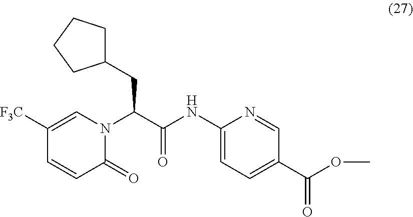

¹H NMR (400 MHz, CDCl₃) δ 9.53 (1H), 8.90 (1H), 8.26-8.29 (1H), 8.17-8.20 (1H), 7.96 (1H), 7.48-7.51 (1H), 6.77-6.80 (1H), 5.80-5.84 (1H), 3.91 (3H), 2.27-2.34 (1 H), 1.92-1.99 (1H), 1.70-1.82 (3H), 1.46-1.62 (4H), 1.13-1.24 (2H); LCMS for C₂₁H₂₂N₃O₄F₃ m/z 438.2 (M+H)⁺.

### Example 28

(S)-6-(3-cyclopentyl-2-(2-oxo-5-(trifluoromethyl)pyridin-1(2H)-yl)propanamido)nicotinic acid mono acetic acid salt (28). Example 28 was made from Example 27 by hydrolyzing the ester to the corresponding acid using procedures outlined in Example 26.

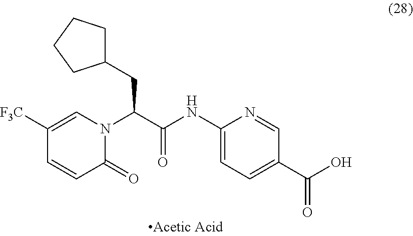

¹H NMR (400 MHz, CD₃OD) δ 8.83 (1H), 8.21-8.24 (2H), 7.80-7.82 (1H), 7.67-7.70 (1 H), 6.65-6.67 (1H), 5.90-5.93 (1H), 2.16-2.21 (2H), 1.89 (3H), 1.48-1.77 (7H), 1.15-1.35 (2H); LCMS for C₂₀H₂₀N₃O₄F₃ m/z 422.5 (M−H)⁺.

### Example 29

(S)-diethyl (5-(3-cyclopentyl-2-(2-oxo-5-(trifluoromethyl)pyridin-1(2H)-yl)propanamido)-pyrazin-2-yl)methylphosphonate (29). Example 29 was made in an analogous manner to Examples 1 and 2, and as described in further detail below.

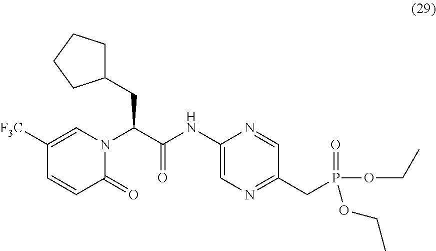

### Intermediate: tert-butyl 5-methylpyrazin-2-ylcarbamate (29a)

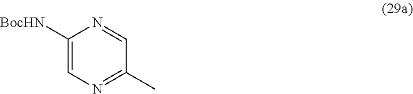

To a stirred solution of 5-methyl-2-carboxylic acid (138 g, 1.0 mol) in dioxane (1 L) was added tert-BuOH (100 mL) and diphenylphosphoryazide (330 g) and the reaction mixture was heated at reflux for 12 hours. The reaction mixture was concentrated to dryness and the residue was purified by flash column chromatography (ethyl acetate/hexanes), then recrystallized from ether to provide tert-butyl 5-methylpyrazin-2-ylcarbamate (29a).

### Intermediate: tert-butyl 5-(bromomethyl)pyrazin-2-ylcarbamate (29b)

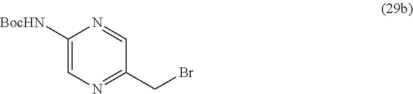

To a solution of tert-butyl 5-methylpyrazin-2-ylcarbamate (29a) (100 g, 0.48 mol) in CCl₄ (40 mL) was added NaHCO₃ followed by N-bromosuccinimide (130 g, 0.57 mol). The resulting mixture was heated to reflux at 80° C. for 12 hours then cooled to room temperature. The reaction mixture was filtered through Celite and the filtrate was concentrated in vacuo. The crude product was chromatographed (SiO₂; 5% EtOAc in CHCl₃) to give tert-butyl 5-(bromomethyl)pyrazin-2-ylcarbamate (29b).

### Intermediate: tert-butyl 5-((diethoxyphosphoryl)methyl)pyrazin-2-ylcarbamate (29c)

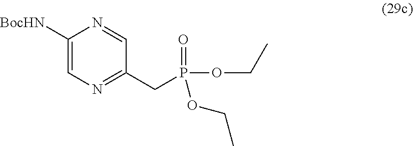

To a stirred solution of tert-butyl 5-(bromomethyl)pyrazin-2-ylcarbamate (29b) (45 g, 0.16 mol) in toluene was added triethylphosphate (0.2 mol). The resulting mixture was refluxed for a day under argon. The resulting mixture was filtered through celite and the filtrate was concentrated under reduced pressure. The residue was purified by flash column chromatography (ethanol-hexanes) to provide tert-butyl 5-(diethoxyphosphoryl)-methyl)pyrazin-2-ylcarbamate (29c) (15 g).

### Intermediate: diethyl (5-aminopyrazin-2-yl)methylphosphonate hydrochloride (29d)

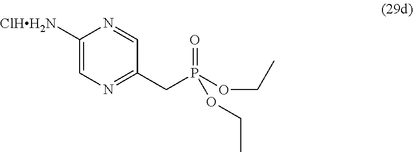

A solution of tert-butyl 5-((diethoxyphosphoryl)methyl)pyrazin-2-ylcarbamate (29c) in 4 N HCl in dioxane was heated at reflux for 6 hours. The reaction mixture was evaporated to dryness. The residue was purified by flash column chromatography to provide diethyl (5-aminopyrazin-2-yl)methylphosphonate hydrochloride (29d). LCMS for C₉H₁₆N₃O₃P m/z 246.1 (M+H)⁺.

Final Preparation: (S)-diethyl (5-(3-cyclopentyl-2-(2-oxo-5-(trifluoromethyl)pyridin-1(2H)-yl)propanamido)pyrazin-2-yl)methylphosphonate (29) was prepared as described in Scheme 3, above.

To a stirred solution of (S)-3-cyclopentyl-2-(2-oxo-5-(trifluoromethyl)pyridin-1(2H)-yl)propanoic acid (167 mg, 0.537 mmol) in CH₂Cl₂ at room temperature was added oxalyl chloride (94 μL, 1.1 mmol) followed by a drop of DMF. After stirring for 90 minutes, the reaction mixture was concentrated under reduced pressure. 1,2-dichloroethane (2 times) was added to the residue and concentrated under reduced pressure to provide (S)-3-cyclopentyl-2-(2-oxo-5-(trifluoromethyl)pyridin-1(2H)-yl)propanoyl chloride. The resulting residue was utilized in the next reaction without further purification.

Diethyl (5-aminopyrazin-2-yl)methylphosphonate hydrochloride (29d) (186 mg, 0.66 mmol) was dissolved in ethanol, followed by the addition of toluene. The mixture was concentrated under reduced pressure while heating at 40° C. The resulting residue was treated with toluene and concentrated to dryness in vacuo. To this residue, a solution of (S)-3-cyclopentyl-2-(2-oxo-5-(trifluoromethyl)pyridin-1(2H)-yl)propanoyl chloride in CH₂Cl₂ followed by pyridine was added. The resulting mixture was stirred at room temperature overnight. The volatiles were removed under reduced pressure. The remaining residue was treated with ethyl acetate, brine, and water. The bilayer was separated and the organics were washed with brine, dried over Na₂SO₄, and filtered. The filtrate was concentrated under reduced pressure and the residue was purified by flash column chromatography on silica gel with ethyl acetate to provide (S)-diethyl (5-(3-cyclopentyl-2-(2-oxo-5-(trifluoromethyl)pyridin-1(2H)-yl)propanamido)-pyrazin-2-yl)methylphosphonate (29). ¹H NMR (400 MHz, CDCl₃) δ 9.48 (1H), 9.34 (1 H), 8.27 (1H), 7.97 (1H), 7.48-7.51 (1H), 6.75-6.78 (1H), 5.81-5.85 (1H), 4.05-4.13 (4H), 3.34-3.39 (2H), 2.28-2.34 (1H), 1.93-1.97 (1H), 1.71-1.82 (3H), 1.48-1.64 (4 H), 1.25-1.29 (6H), 1.15-1.26 (2H); LCMS for C₂₃H₃₀N₄O₅F₃P m/z 531.2 (M+H)⁺.

### Example 30

### (S)—N-(1-benzyl-1H-pyrazol-3-yl)-3-cyclopentyl-2-(2-oxo-4-(trifluoromethyl)pyridin-1(2H)-yl)propanamide (30)

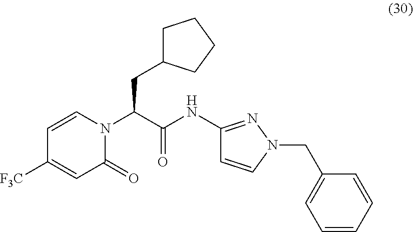

¹H NMR (400 MHz, CDCl₃) δ 10.46 (1H), 7.96-7.98 (1H), 7.39 (1H), 7.22-7.33 (4H), 7.15-7.17 (2H), 6.68 (1H), 6.37-6.39 (1H), 6.06-6.10 (1H), 5.17 (2H), 2.19-2.24 (1H), 1.63-1.94 (4H), 1.45-1.60 (4H), 1.12-1.23 (2H); LCMS for C₂₄H₂₅N₄O₂F₃ m/z 459.2 (M+H)⁺.

### Example 31

### (S)-3-cyclopentyl-N-(1-(2-hydroxy-2-methylpropyl)-1H-pyrazol-3-yl)-2-(2-oxo-5-(trifluoromethyl)pyridin-1(2H)-yl)propanamide (31)

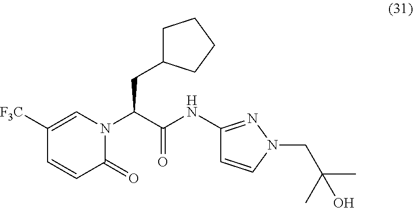

¹H NMR (400 MHz, CDCl₃) δ 9.75 (1H), 8.10 (1H), 7.46-7.49 (1H), 7.28 (1H), 6.80-6.82 (1H), 6.66 (1H), 5.85-5.89 (1H), 3.93 (2H), 3.70 (1H), 2.22-2.29 (1H), 1.87-1.94 (1H), 1.68-1.81 (3H), 1.45-1.62 (4H), 1.09-1.20 (8H); LCMS for C₂₁H₂₇N₄O₃F₃ m/z 441.2 (M+H)⁺.

### Example 32

### (S)-3-cyclopentyl-N-(1-(2-hydroxy-2-methylpropyl)-1H-pyrazol-3-yl)-2-(2-oxo-4-(trifluoromethyl)pyridin-1(2H)-yl)propanamide (32)

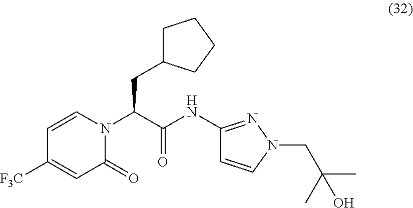

¹H NMR (400 MHz, CDCl₃) δ 0.96-1.22 (8H), 1.41-1.67 (5H), 1.71-1.85 (2H), 1.95 (1H), 2.25 (1H), 3.42 (1H), 3.80-3.99 (2H), 5.88 (1H), 6.40 (1H), 6.66 (1H), 7.12 (1 H), 7.29 (1H), 7.85 (1H), 9.72 (1H); LCMS for C₂₁H₂₇N₄O₃F₃ m/z 441.1 (M+H)⁺.

### Example 33

### (S)-3-cyclopentyl-N-(5-(hydroxymethyl)pyridin-2-yl)-2-(2-oxo-5-(trifluoromethyl)pyridin-1(2H)-yl)propanamide (33)

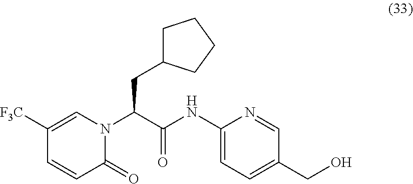

¹H NMR (400 MHz, CDCl₃) δ 9.79 (1H), 8.36 (1H), 8.10 (1H), 8.03 (1H), 7.70-7.73 (1 H), 7.46-7.49 (1H), 6.73 (1H), 5.76-5.80 (1H), 5.29 (2H), 2.19-2.26 (1H), 1.88-1.95 (1H), 1.40-1.76 (7H), 1.22-1.26 (1H), 1.06-1.14 (2H); LCMS for C₂₀H₂₂N₃O₃F₃ m/z 410.1 (M+H)⁺.

### Example 34

(S)-6-(3-cyclopentyl-2-(2-oxo-4-(trifluoromethyl)pyridin-1(2H)-yl)propanamido)nicotinic acid (34). Example 34 was prepared in a manner analogous to example 28.

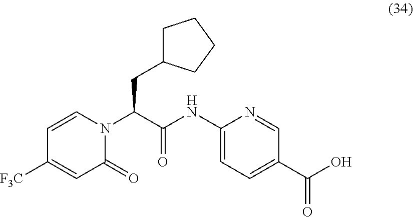

¹H NMR (400 MHz, CDCl₃) δ 1.02 (1H), 1.27-1.71 (8H), 1.96-2.08 (1H), 2.23 (1H), 5.78-5.89 (1H), 6.52 (1H), 6.81 (1H), 8.05 (1H), 8.23 (1H), 8.83 (1H), 11.54 (1H), 13.18 (1H); LCMS for C₂₀H₂₀N₃O₄F₃ m/z 424.0 (M+H)⁺.

### Example 35

### (S)-3-cyclopentyl-2-(4-(difluoromethyl)-6-oxopyrimidin-1(6H)-yl)-N-(5-methylpyrazin-2-yl)propanamide (35)

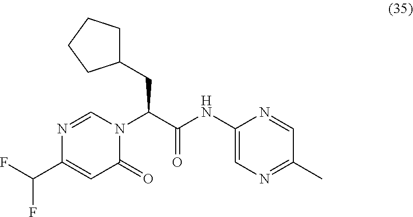

¹H NMR (400 MHz, DMSO-d₆) δ 11.40 (1H), 9.07 (1H), 8.73 (1H), 8.29 (1H), 6.80 (1 H), 6.64 (s, 1H), 5.69 (1H), 2.41 (3H), 2.31 (1H), 1.92-2.14 (1H), 1.15-1.70 (8H), 0.89-1.12 (1H); LCMS for C₁₈H₂₁F₂N₅O₂ m/z 378.2 (M+H)⁺.

### Example 36

### (S)-3-cyclopentyl-2-(4-(difluoromethyl)-6-oxopyrimidin-1(6H)-yl)-N-(5-methylpyridin-2-yl)propanamide (36)

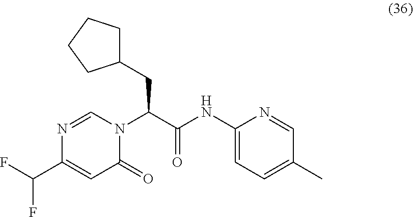

¹H NMR (400 MHz, DMSO-d₆) δ 11.14 (1H), 8.70 (1H), 8.14 (1H), 7.83 (1H), 7.57 (1 H), 6.68 (1H), 5.72 (1H), 2.20 (3H), 1.94-2.11 (1H), 1.24-1.73 (8H), 0.94-1.12 (3 H); LCMS for C₁₉H₂₂F₂N₄O₂ m/z 378.2 (M+H)⁺.

### Example 37

### (S)-6-(3-cyclohexyl-2-(2-oxo-4-(trifluoromethyl)pyridin-1(2H)-yl)propanamido)nicotinic acid (37)

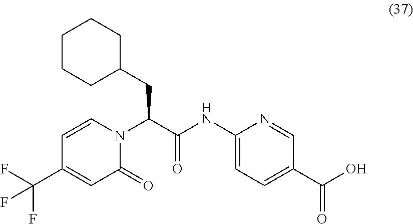

¹H NMR (400 MHz, CD₃OD) δ 8.90 (1H), 8.30 (1H), 8.15 (1H), 8.05 (1H), 6.85 (1H), 6.61 (1H), 5.98 (1H), 2.04-2.12 (2H), 1.60-1.85 (5H), 0.98-1.36 (9H).

### Example 38

### (S)—N-(5-methylpyridin-2-yl)-2-(2-oxo-5-(trifluoromethyl)pyridin-1(2H)-yl)-3-(tetrahydro-2H-pyran-4-yl)propanamide (38)

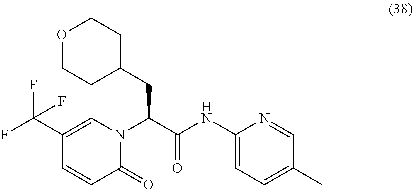

### Intermediate: 3,6-dihydro-2H-pyran-4-yl trifluoromethanesulfonate (38a)

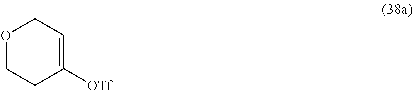

Under argon, diisopropylamine (66.8 g (92.5 mL), 0.66 mol) was dissolved in THF (1 L) and cooled to −5° C. in an ice/methanol bath. Over 30 minutes, n-butyllithium (2.34 M, 290 mL, 0.66 mol) was added while maintaining the temperature below 1° C. The mixture was stirred at about 0° C. to about −5° C. for 15 minutes and cooled to −72° C. with an acetone and dry ice bath. Dihydro-2H-pyran-4(3H)-one was added slowly over 15 minutes while maintaining the temperature at −78° C. for 1 hour. N-phenyl-bis-(trifluoromethyl sulfonimide) was suspended in THF (500 mL) and added slowly to the mixture while maintaining a temperature below −60° C. The mixture was left stirring in the cooling bath, warming to room temperature overnight. The mixture was concentrated under reduced pressure. The residues were slurried in hexane at 50° C. (1 L and 250 mL), the liquors were concentrated under reduced pressure to afford (38a). ¹H NMR (CDCl₃, 300 MHz) δ 5.74 (1H); 4.19 (2H); 3.80 (2H); 2.39 (2H).

### Intermediate: (R)-methyl 2-(tert-butoxycarbonyl)-3-(3,6-dihydro-2H-pyran-4-yl)propanoate (38b)

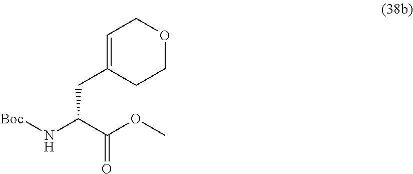

In rigorously anaerobic conditions, zinc dust (72.7 g, 1.11 mol) was suspended in anhydrous DMF (100 mL), and to the stirred solution, trimethylsilyl chloride (23 mL 0.18 mol) was added (exotherm to 55° C.). The mixture was stirred for 20 minutes, during which time the supernatant became brown in color. The mixture was allowed to settle, and the supernatant decanted off using vacuum. The activated zinc powder was washed with DMF (4×50 mL), until the supernatant solvent became colorless.

(R)-methyl 2-(tert-butoxycarbonylamino)-3-iodopropanoate (85 g, 0.26 mol) was dissolved in DMF under argon, added in one portion to the activated zinc powder and stirred briskly. After approximately 5 minutes, the mixture self heated rapidly (21-30° C. over about 15 seconds). The stirring was stopped and the cooling bath immediately applied, allowing the exothermic reaction to be ceased at 50° C. As the temperature subsided, the cooling bath was removed and the mixture stirred at ambient temperature for 20 minutes and allowed to settle. The supernatant was syringed into a pre-prepared solution of Intermediate (38a) (60 g, 0.26 mol) and PdCl₂(PPh₃)₂ (5.44 g, 7.75 mmol). The metallic solids were washed with DMF (30 mL) and the washings added to the triflate/catalyst mixture, which was stirred at 50° C. overnight. The solution was concentrated under reduced pressure and the crude product slurried in water (500 mL) and 20% ethyl acetate in hexane (500 mL). The mixture was filtered and partitioned, and the aqueous layer re-extracted with 20% ethyl acetate in hexane (500 mL). The combined organic phases were washed with brine (500 mL), dried over MgSO₄, and concentrated under reduced pressure. The semi-crude product was obtained as a free running red-brown oil (81 g), which was purified twice by dry-flash chromatography (SiO₂, ethyl acetate and hexanes, 0 to 100%) followed by carbon treatment in 10% ethyl acetate/hexane to afford (38b): ¹H NMR (CDCl₃, 300 MHz): δ 5.50 (1H), 4.95 (1H), 4.40 (1H), 4.10 (2H), 3.77 (2H), 3.73 (3H), 2.50 (1H), 2.31 (1H), 2.07 (2H), 1.43 (9H).

### Intermediate: (R)-methyl 2-(tert-butoxycarbonyl)-3-(tetrahydro-2H-pyran-4-yl)propanoate (38c)

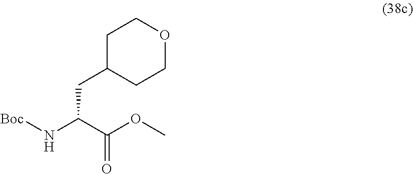

In a stainless steel autoclave, 22.83 g (80.0 mmol) of Intermediate (38b) was dissolved in methanol (150 mL) to which was added 5% Pd/C (2.3 g) as a slurry in toluene (10 mL). The autoclave was charged to 20 bar with hydrogen and the reaction mixture was stirred for 2 hours at room temperature. The mixture was filtered through celite and the filtrates concentrated under reduced pressure to afford (38c). The product was used in the next step without further purification. ¹H NMR (CDCl₃, 300 MHz): δ 4.92 (1H), 4.38 (1H), 3.92 (2H), 3.73 (3H), 3.35 (2H), 1.5-1.8 (4H), 1.43 (9H), 1.2-1.4 (2H).

### Intermediate: (R)-2-amino-3-(tetrahydro-2H-pyran-4-yl)propanoic acid (38d)

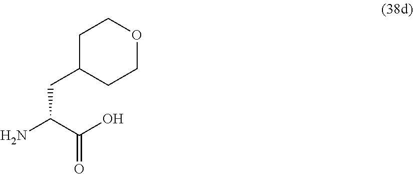

Intermediate (38c) (22.9 g, 80.0 mmol) was suspended in 6N aqueous HCl (200 mL) and heated at 100° C. overnight. The mixture was cooled to room temperature and extracted with 20% ethyl acetate/hexane (100 mL) to remove any unwanted organics. The aqueous phase was concentrated under reduced pressure and co-distilled with toluene (2×200 mL) to afford the HCl salt of (38d), giving a yield of 17.9 g; 108% (off-white powder, presumed damp with water or toluene). ¹H NMR (DMSO-d₆, 300 MHz) δ 8.49 (3H), 3.79 (3H), 3.19 (2H), 2.44 (1H), 1.4-1.9 (5H), 1.12 (2H).

Secondly, The HCl salt of (38d) (11.6 g, 55.3 mmol) and isobutylene oxide (5.33 mL) were suspended in DMF (120 mL) in 4 Anton Paar 30 mL microwave vials. The mixtures were reacted at 100° C. for 1 hour and allowed to cool. The mixtures were washed out of the vials with ethyl acetate (50 mL each), combined and stirred briskly in further ethyl acetate (total volume 500 mL) for 10 minutes, during which time a thick cream-colored suspension formed. The solids were filtered off, broken up with a spatula and dried under vacuum oven at 50° C. overnight to afford (38d).

### Intermediate: (R)-2-hydroxy-3-(tetrahydro-2H-pyran-4-yl)propanoic acid (38e)

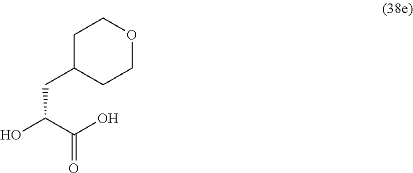

Compound (38d) (7.68 g, 44.3 mmol) was dissolved in 1N H₂SO₄ (140 mL) and cooled to 0° C. under argon. NaNO₂ (4.6 g, 66.45 mmol) as a solution in water (25 mL) was introduced drop-wise under the surface of the mixture and the whole stirred overnight. The mixture was extracted with ethyl acetate (100 mL). The aqueous phase was extracted with further ethyl acetate (5×100 mL). The aqueous phase was cooled to 0° C. under argon and re-dosed with concentrated H₂SO₄ (3.5 mL) and NaNO₂ (4.6 g, 66.45 mmol) as a solution in water (25 mL) and stirred overnight. The mixture was extracted with ethyl acetate (6×100 mL), re-dosed as above, stirred overnight and finally extracted a third time with ethyl acetate (6×100 mL). All 1800 mL of organics were combined and stripped to afford (38e) with a yield of 7.0 g (91%) as an orange oil. ¹H NMR (CD₃OD, 300 MHz): δ 4.20 (1H), 3.92 (2H), 3.39 (2H), 1.7 (2H), 1.6 (2H), 1.27 (2H).

### Intermediate: (R)-methyl 2-hydroxy-3-(tetrahydro-2H-pyran-4-yl)propanoate (38f)

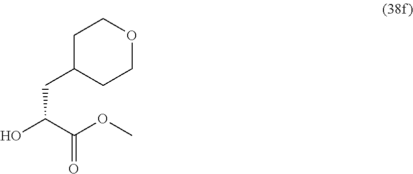

Compound (38e) (9.0 g, 51 mmol) was dissolved in methanol (100 mL) and stirred. HCl was sparged in to the mixture for 15 minutes (exothermic 20 to 65° C.) and the whole was refluxed for 7 hours and allowed to cool. The mixture was stripped to approximately ⅓ volume, diluted with water (100 mL) and extracted with ethyl acetate (2×100 mL). The organics were stripped and the crude product purified by dry-flash chromatography (SiO₂, ethyl acetate and hexanes, 0 to 100%) to 3.8 g of Intermediate (38f). The aqueous phase was re-extracted with ethyl acetate (2×200 mL), stripped, and re-purified to a further 1.2 g of Intermediate (38f): ¹H NMR (CDCl₃, 300 MHz): δ 4.24 (1H), 3.95 (2H), 3.78 (3H), 3.39 (2H), 2.73 (1H), 1.83 (1H), 1.52-1.75 (4H), 1.22-1.42 (1H).

### Intermediate: (R)-methyl 3-(tetrahydro-2H-pyran-4-yl)-2-(trifluoromethylsulfonyloxy)-propanoate (38 g)

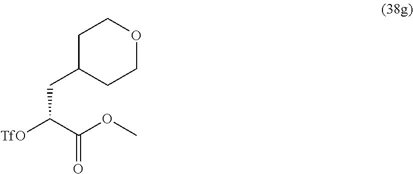

Compound (38f), (1.21 g, 6.43 mmol) was dissolved in anhydrous dichloromethane (60 mL) under nitrogen. The mixture was stirred in an ice bath, and lutidine (1.6 mL) was added. Triflic anhydride (1.95 mL, 11.6 mmol) was added drop-wise, and the reaction was stirred for 60 minutes. The reaction mixture was diluted with methyl tert-butyl ether, and washed 3-times with 3:1 brine/1 N HCl. The organic layer was dried over MgSO₄, filtered, evaporated, and dried under high vacuum to afford (38 g), which was utilized in the following reaction without further purification.

Final compound: ((S)—N-(5-methylpyridin-2-yl)-2-(2-oxo-5-(trifluoromethyl)pyridin-1(2H)-yl)-3-(tetrahydro-2H-pyran-4-yl)propanamide (38). Intermediate 38 g was converted to final product 38 in a manner analogous to examples 1 and 2.

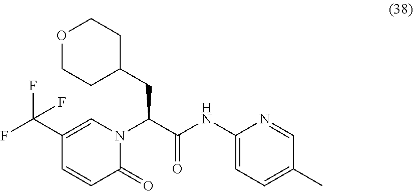

¹H NMR (400 MHz, CDCl₃) δ 8.98 (1H), 8.11 (1H), 7.93-7.98 (2H), 7.46-7.51 (2H), 6.71-6.73 (1H), 5.81-5.85 (1H), 3.90-3.93 (2H), 3.26-3.35 (2H), 2.27 (3H), 2.19-2.26 (1H), 1.81-1.88 (1H), 1.63-1.68 (2H), 1.27-1.47 (3H); LCMS for C₂₀H₂₂N₃O₃F₃ m/z 410.5 (M−H)⁺.

Examples 39-43 were prepared in a manner analogous to example 38 using appropriate starting materials.

### Example 39

### (S)—N-(5-methylpyrazin-2-yl)-2-(2-oxo-5-(trifluoromethyl)pyridin-1(2H)-yl)-3-(tetrahydro-2H-pyran-4-yl)propanamide (39)

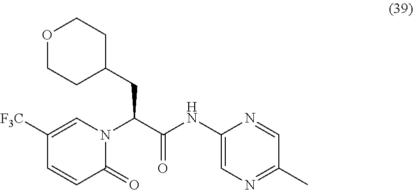

¹H NMR (400 MHz, CDCl₃) δ 9.34 (1H), 9.25 (1H), 8.07 (1H), 7.95 (1H), 7.48-7.51 (1 H), 6.74-6.76 (1H), 5.90-5.94 (1H), 3.90-3.93 (2H), 3.26-3.35 (2H), 2.49 (3H), 2.22-2.30 (1H), 1.83-1.90 (1H), 1.65-1.68 (2H), 1.24-1.51 (3H); LCMS for C₁₉H₂₁N₄O₃F₃ m/z 411.1 (M+H)⁺.

### Example 40

### (S)—N-(1-methyl-1H-pyrazol-3-yl)-2-(2-oxo-5-(trifluoromethyl)pyridin-1(2H)-yl)-3-(tetrahydro-2H-pyran-4-yl)propanamide (40)

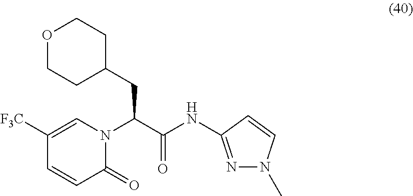

¹H NMR (400 MHz, CDCl₃) δ 9.24 (1H), 7.99 (1H), 7.45-7.49 (1H), 7.23-7.25 (1H), 6.77-6.79 (1H), 6.57 (1H), 5.83-5.87 (1H), 3.90-3.95 (2H), 3.78 (3H), 3.26-3.35 (2H), 2.19-2.27 (1H), 1.77-1.84 (1H), 1.64-1.67 (2H), 1.42-1.50 (1H), 1.28-1.39 (2H); LCMS for C₁₈H₂₁N₄O₃F₃ m/z 399.04 (M+H)⁺.

### Example 41

### (S)—N-(5-methylpyridin-2-yl)-2-(2-oxo-4-(trifluoromethyl)pyridin-1(2H)-yl)-3-(tetrahydro-2H-pyran-4-yl)propanamide (41)

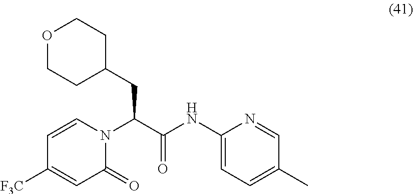

¹H NMR (400 MHz, CDCl₃) δ 9.34 (1H), 8.11 (1H), 7.97-7.99 (1H), 7.74-7.76 (1H), 7.47-7.50 (1H), 7.09 (1H), 6.40-6.42 (1H), 5.93-5.96 (1H), 3.89-3.93 (2H), 3.26-3.35 (2H), 2.27 (3H), 2.16-2.23 (1H), 1.84-1.91 (1H), 1.64-1.70 (2H), 1.29-1.48 (3H); LCMS for C₂₀H₂₂N₃O₃F₃ m/z 410.5 (M+H)⁺.

### Example 42

### (S)—N-(5-methylpyrazin-2-yl)-2-(2-oxo-4-(trifluoromethyl)pyridin-1(2H)-yl)-3-(tetrahydro-2H-pyran-4-yl)propanamide (42)

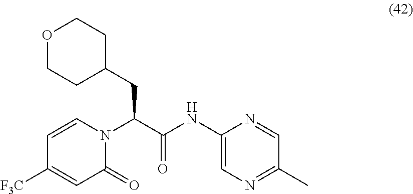

¹H NMR (400 MHz, CDCl₃) δ 9.85 (1H), 9.29 (1H), 8.09 (1H), 7.81-7.83 (1H), 7.18 (1H), 6.45-6.47 (1H), 6.07-6.11 (1H), 3.91-3.94 (2H), 3.28-3.36 (2H), 2.50 (3H), 2.19-2.27 (1H), 1.87-1.94 (1H), 1.66-1.71 (2H), 1.32-1.51 (3H); LCMS for C₁₉H₂₁N₄O₃F₃ m/z 411.5 (M+H)⁺.

### Example 43

### (S)—N-(1-methyl-1H-pyrazol-3-yl)-2-(2-oxo-4-(trifluoromethyl)pyridin-1(2H)-yl)-3-(tetrahydro-2H-pyran-4-yl)propanamide (43)

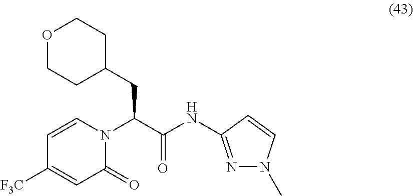

¹H NMR (400 MHz, CDCl₃) δ 10.58 (1H), 8.01-8.02 (1H), 7.53 (1H), 7.25-7.27 (1H), 6.65 (1H), 6.43-6.45 (1H), 6.19-6.22 (1H), 3.92-3.95 (2H), 3.81 (3H), 3.32-3.38 (2 H), 2.20-2.25 (1H), 1.81-1.86 (1H), 1.74-1.77 (2H), 1.49-1.56 (1H), 1.33-1.41 (2H); LCMS for C₁₈H₂₁N₄O₃F₃ m/z 399.04 (M+H)⁺.

### Biochemical Data

Full-length glucokinase (beta cell isoform) was His-tagged at N-terminus and purified by a Ni column followed by size exclusion chromatography. A 320 mL column was packed in house using Pharmacia Superdex75 preparation grade resin. Glucose was obtained from Calbiochem (San Diego, Calif.) and other reagents were purchased from Sigma (St. Louis, Mo.).

All assays were performed in a Corning 384-well plate using Spectramax PLUS spectrophotometer (Molecular Devices, Sunnyvale, Calif.) at room temperature. The final assay volume was 40 μL. The buffer conditions used in this assay were as follows: 50 mM HEPES, 5 mM glucose, 2.5 mM ATP, 3.5 mM MgCl₂, 0.7 mM NADH, 2 mM dithiothreitol, 1 Unit/mL PK/LDH, 0.2 mM phosphoenolpyruvate, and 25 mM KCl. The buffer pH was 7.1. The test compound in DMSO solution was added to the buffer and mixed by a plate shaker for 7.5 minutes. The final concentration of DMSO introduced into the assay was 0.25%.

Glucokinase was added to the buffer mixture to initiate the reaction in the presence and absence of compound. The reaction was monitored by absorbance at 340 nm due to the depletion of NADH. The initial reaction velocity was measured by the slope of a linear time course of 0-300 seconds. The percentage of activation was calculated by the following equation:

% Activation=(Va/Vo−1)×100;

wherein each of Va and Vo is defined as the initial reaction velocity in the presence and absence of the tested compound, respectively.

To determine the EC₅₀ (half maximal effective concentration) and % maximum activation, compounds were serially diluted in DMSO by 3-fold. The GK activities were measured as a function of compound concentrations. The data were fitted to the equation below to obtain the EC₅₀ and % max activation values:

Va/Vo=1+(% max activation/100)/(1+EC₅₀/compound concentration)

### Beta Cell Glucokinase His-Tag Purification

### Growth and Induction Conditions:

BL21(DE3) cells containing pBCGK (C or N His) vector were grown at 37° C. (in 2XYT) until the OD600 was between 0.6-1.0. Expression was induced by addition of isopropylthiogalactoside (IPTG) to a final concentration of 0.1-0.2 mM to the cells which were then incubated overnight at 23° C. The next day, cells were harvested via centrifugation at 5000 rpm for 15 minutes at 4° C. The cell pellet was stored at −80° C. for future purification.

### Purification:

A Ni-NTA column (15-50 mL) was used for separation. Two buffers were prepared, 1) a lysis/nickel equilibration and wash buffer and 2) a nickel elution buffer. The lysis/equilibration/wash buffer was prepared as such: 25 mM Hepes buffer at pH 7.5, 250 mM NaCl, 20 mM imidazole, and 14 mM β-mercaptoethanol as final concentrations. The elution buffer was prepared as such: 25 mM Hepes at pH 7.5, 250 mM NaCl, 400 mM imidazole, and 14 mM β-mercaptoethanol as final concentrations. The buffers were each filtered with a 0.22 μm filter prior to use. The cell pellet (1 L culture) was resuspended in 300 mL of the lysis/equilibration buffer. The cells were then lysed (3 times) with a microfluidizer (Microfluidics Corporation, Model 110Y). The slurry was centrifuged with an ultracentrifuge (Beckman Coulter, Model LE-80K) at 40,000 rpm for 45 minutes at 4° C. The supernatant was transferred to a chilled flask. A volume of 20 μl was saved for gel analysis. The AKTA (Pharmacia purification system) prime lines were purged with lysis/equilibration buffer. The Ni-NTA column was equilibrated with 200 mL of the lysis/equilibration buffer at a flow rate of 5 mL/minute. The supernatant was loaded over the column at 4 mL/minute and the flow-through was collected in a flask. The unbound proteins were washed with lysis/equilibration buffer at a flow rate of 5 mL/minute until UV reaches the baseline. The protein was then eluted from the column with the imidazole elution buffer via imidazole gradient 20 mM to 400 mM over 320 mL. The column was then stripped of any additional protein with 80 mL of the elution buffer. The elution fractions were each 8 mL, for a total yield of 50 samples. Fractions were analyzed by SDS-PAGE and the fractions containing protein of interest were pooled and concentrated to 10 mL using ultrafiltration cell with 10,000 MWCO membrane (Millipore) under nitrogen gas (60 psi). Protein was further purified by SEC using Sudex75 (320 mL, Pharmacia). SEC was equilibrated with 450 mL sizing buffer containing 25 mM Hepes pH 7.0, 50 mM NaCl, and 5 mM dithiothreitol. Concentrated protein was then loaded over SEC and elution with 400 mL sizing buffer was performed overnight at 0.5 mL/minute. The elution fractions were 5 mL each. The fractions were analyzed by SDS-PAGE and protein containing fractions were pooled. Concentration was measured using Bradford Assay/BSA Standard. Purified protein was stored in small aliquots at −80° C.

### Biological Data

Table 1. EC₅₀ (μM) and Percent Maximum Activation Data obtained from the biological procedures as defined above. Values are presented as a range where sample size (N) is >1.

TABLE 1

| Example | EC₅₀ (μM) | Maximum Activation (%) | N |
|---|---|---|---|
| 1 | 5.0-10.5 | 76-100 | 4 |
| 2 | >100 | 0 | 1 |
| 3 | 1.0-2.5 | 55-79 | 5 |
| 4 | 4.6-22.4 | 72-106 | 4 |
| 5 | >100 | 0 | 1 |
| 6 | 0.2-0.8 | 88-105 | 5 |
| 7 | 1.6-3.7 | 118-152 | 6 |
| 8 | 1.7-3.2 | 110-128 | 4 |
| 9 | 1.10-1.40 | 95-106 | 4 |
| 10 | 1.5-2.1 | 121-148 | 4 |
| 11 | 0.7-1.3 | 108-141 | 4 |
| 12 | 13.6-20.8 | 109-110 | 2 |
| 13 | 14 | 106 | 1 |
| 14 | 5.8 | 58 | 1 |
| 15 | 9.4 | 92 | 1 |
| 16 | 2.5 | 125 | 1 |
| 17 | 0.4 | 57 | 1 |
| 18 | 9.3 | 103 | 1 |
| 19 | 0.5 | 89 | 1 |
| 20 | 1.2 | 114 | 1 |
| 21 | 0.3 | 113 | 1 |
| 22 | 20.2 | 130 | 1 |
| 23 | 2.6 | 77 | 1 |
| 24 | 27.1 | 106 | 1 |
| 25 | 2.9 | 84 | 1 |
| 26 | 11.1 | 116 | 1 |
| 27 | 0.2 | 84 | 1 |
| 28 | 0.5 | 147 | 1 |
| 29 | 5.7 | 141 | 1 |
| 30 | 4.2 | 58 | 1 |
| 31 | 1.1-2.0 | 172-194 | 2 |
| 32 | 6.9-7.5 | 87-90 | 2 |
| 33 | 0.3-0.5 | 85-159 | 3 |
| 34 | 1.8-2.7 | 100-102 | 2 |
| 35 | 45 | 144 | 1 |
| 36 | 8.1 | 106 | 1 |
| 37 | 1.5-3.5 | 92-101 | 2 |
| 38 | 2.9-4.6 | 57-70 | 2 |
| 39 | >100 | — | 1 |
| 40 | 54 | 121 | 1 |
| 41 | >100 | — | 1 |
| 42 | >100 | — | 1 |
| 43 | >100 | — | 1 |

We claim:

1. A compound of Formula (1A) 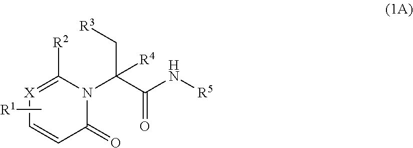
X is carbon or nitrogen;
R¹ is —CF₂R^{a} where R^{a} is H, F, or (C₁-C₆)alkyl;
R² is H, halo, CF₃, (C₁-C₆)alkyl, or (C₁-C₃)alkoxy;
R³ is a chemical moiety selected from the group consisting of (C₃-C₆)cycloalkyl, 5- to 6-membered heterocycle, 5- to 6-membered heteroaryl, and phenyl, wherein said heterocycle or said heteroaryl contains one to three heteroatoms each independently N, O, or S, and where said moiety is optionally substituted with one to three substituents each independently halo, (C₁-C₆)alkyl, (C₁-C₆)alkoxy, CF₃, or cyano;
R⁴ is H or (C₁-C₆)alkyl; and
R⁵ is a chemical moiety selected from the group consisting of a 5- to 6-membered heteroaryl and quinolinyl, wherein said heteroaryl contains one to three heteroatoms each independently N, O, or S, and where said moiety is optionally substituted with one to three R⁶ substituents each independently (C₁-C₆)alkyl, CF₃, cyano, (C₁-C₆)alkoxy, halo, amino, (C₁-C₃)alkylamino, di-(C₁-C₃)alkylamino, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, and where the aryl of said arylalkyl is optionally substituted with one to three substituents each independently (C₁-C₆)alkyl, CF₃, cyano, (C₁-C₆)alkoxy, halo, carboxy, amino, (C₁-C₃)alkylamino, or di-(C₁-C₃)alkylamino;
or a pharmaceutically acceptable salt thereof.

2. The compound of claim 1 wherein
R² is H, F, Cl, CF₃, methyl, ethyl, methoxy, or ethoxy; and
R³ is a chemical moiety selected from the group consisting of (C₃-C₆)cycloalkyl and 5- to 6-membered heterocycle, wherein said heterocycle contains one to three heteroatoms each independently N, O, or S, and where said moiety is optionally substituted with one to three substituents each independently halo, (C₁-C₆)alkyl, (C₁-C₆)alkoxy, CF₃, or cyano;
or a pharmaceutically acceptable salt thereof.

3. The compound of claim 2 wherein
R³ is a chemical moiety selected from the group consisting of cyclobutyl, cyclopentyl, and tetrahydropyranyl, wherein said moiety is optionally substituted with one to three substituents each independently halo, (C₁-C₆)alkyl, (C₁-C₆)alkoxy, CF₃, or cyano; and
R⁴ is H, methyl, or ethyl;
or a pharmaceutically acceptable salt thereof.

4. The compound of claim 3 wherein
R⁵ is a chemical moiety selected from the group consisting of pyrrolyl, pyrazolyl, imidazolyl, isoxazolyl, oxazolyl, isothiazolyl, thiazolyl, pyridinyl, pyridazinyl, pyrimidinyl, pyrazinyl, and quinolinyl, wherein said moiety is optionally substituted with one to three R⁶ substituents each independently (C₁-C₆)alkyl, CF₃, cyano, (C₁-C₆)alkoxy, halo, amino, (C₁-C₃)alkylamino, di-(C₁-C₃)alkylamino, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, and where the aryl of said arylalkyl is optionally substituted with one to three substituents each independently (C₁-C₆)alkyl, CF₃, cyano, (C₁-C₆)alkoxy, halo, carboxy, amino, (C₁-C₃)alkylamino, or di-(C₁-C₃)alkylamino;
or a pharmaceutically acceptable salt thereof.

5. The compound of claim 4 wherein
R⁴ is H; and
R⁵ is a chemical moiety selected from the group consisting of pyrazolyl, isoxazolyl, thiazolyl, pyridinyl, pyrimidinyl, pyrazinyl, and quinolinyl, wherein said moiety is optionally substituted with one to three R⁶ substituents each independently (C₁-C₆)alkyl, CF₃, cyano, (C₁-C₆)alkoxy, halo, amino, (C₁-C₃)alkylamino, di-(C₁-C₃)alkylamino, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, and where the aryl of said arylalkyl is optionally substituted with one to three substituents each independently (C₁-C₆)alkyl, CF₃, cyano, (C₁-C₆)alkoxy, halo, carboxy, amino, (C₁-C₃)alkylamino, or di-(C₁-C₃)alkylamino;
or a pharmaceutically acceptable salt thereof.

6. The compound of claim 5 wherein
R³ is cyclopentyl or tetrahydropyranyl; and
R⁵ is a chemical moiety selected from the group consisting of 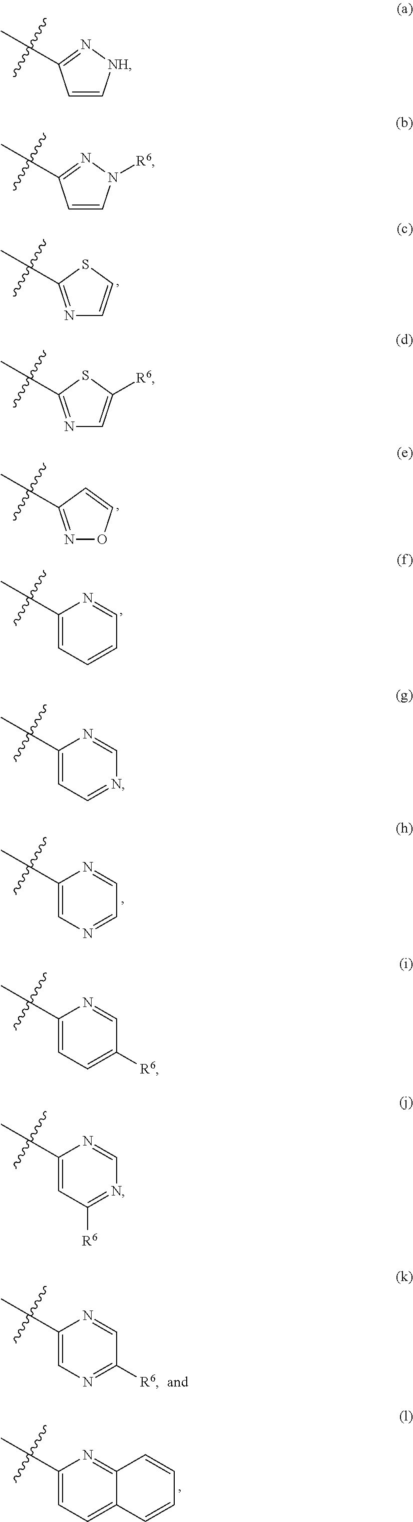
wherein R⁶ is (C₁-C₆)alkyl, CF₃, (C₁-C₆)alkoxy, halo, —CH₂P(O)(OR⁷)(OR⁸), —C(O)OR⁷, —CH₂C(O)OR⁷, or aryl(C₁-C₆)alkyl, where R⁷ and R⁸ are each independently H or (C₁-C₆)alkyl, and where the aryl of said arylalkyl is optionally substituted with one to three substituents each independently (C₁-C₆)alkyl, CF₃, cyano, (C₁-C₆)alkoxy, halo, carboxy, amino, (C₁-C₃)alkylamino, or di-(C₁-C₃)alkylamino;
or a pharmaceutically acceptable salt thereof.

7. The compound of claim 6 wherein
X is carbon; and
R⁶ is methyl, ethyl, methoxy, CF₃, methoxy, ethoxy, halo, —CH₂P(O)(OR⁷)(OR⁸), —C(O)OR⁷, —CH₂C(O)OR⁷, or benzyl, where R⁷ and R⁸ are each independently selected from H, methyl, or ethyl;
or a pharmaceutically acceptable salt thereof.

8. A compound selected from the group consisting of
(S)-3-cyclopentyl-N-(5-methylpyrazin-2-yl)-2-(2-oxo-4-(trifluoromethyl)pyridin-1(2H)-yl)propanamide;
(S)-3-cyclopentyl-N-(5-methylpyridin-2-yl)-2-(2-oxo-3-(trifluoromethyl)pyridin-1(2H)-yl)propanamide;
(S)-3-cyclopentyl-N-(5-methylpyridin-2-yl)-2-(2-oxo-4-(trifluoromethyl)pyridin-1(2H)-yl)propanamide;
(S)-3-cyclopentyl-N-(1-methyl-1H-pyrazol-3-yl)-2-(2-oxo-4-(trifluoromethyl)pyridin-1(2H)-yl)propanamide;
(S)-3-cyclopentyl-N-(5-methylpyrazin-2-yl)-2-(2-oxo-3-(trifluoromethyl)pyridin-1(2H)-yl)propanamide;
(S)-3-cyclopentyl-N-(5-methylpyridin-2-yl)-2-(2-oxo-5-(trifluoromethyl)pyridin-1(2H)-yl)propanamide;
(S)-3-cyclopentyl-N-(5-methylpyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyrazin-2-yl)propanamide;
(S)-3-cyclopentyl-2-(2-oxo-5-(trifluoromethyl)pyridin-1(2H)-yl)-N-(pyrimidin-4-yl)propanamide;
(S)-3-cyclopentyl-N-(1-ethyl-1H-pyrazol-3-yl)-2-(2-oxo-5-(trifluoromethyl)pyridin-1(2H)-yl)propanamide;
(S)—N-(1-benzyl-1H-pyrazol-3-yl)-3-cyclopentyl-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyrimidin-2-yl)propanamide;
(S)-3-cyclopentyl-2-(2-oxo-4-(trifluoromethyl)pyridin-1(2H)-yl)-N-(pyrazin-2-yl)propanamide;
(S)-3-cyclopentyl-2-(2-oxo-4-(trifluoromethyl)pyridin-1(2H)-yl)-N-(pyrimidin-4-yl)propanamide;
(S)-3-cyclopentyl-N-(1-ethyl-1H-pyrazol-3-yl)-2-(2-oxo-4-(trifluoromethyl)pyridin-1(2H)-yl)propanamide;
(S)-3-cyclopentyl-N-(1-methyl-1H-pyrazol-3-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(5-(trifluoromethyl)pyridin-2-yl)propanamide;
(S)-3-cyclopentyl-N-(isoxazol-3-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(quinolin-2-yl)propanamide;
(S)-3-cyclopentyl-N-(5-methoxypyrazin-2-yl)-2-(2-oxo-5-(trifluoromethyl)pyridin-1(2H)-yl)propanamide;
(S)-3-cyclopentyl-2-(2-oxo-5-(trifluoromethyl)pyridin-1(2H)-yl)-N-(pyridin-2-yl)propanamide;
(S)-ethyl 2-(2-(3-cyclopentyl-2-(2-oxo-5-(trifluoromethyl)pyridin-1(2H)-yl)propanamido)thiazol-5-yl)acetate;
(S)-2-(2-(3-cyclopentyl-2-(2-oxo-5-(trifluoromethyl)pyridin-1(2H)-yl)propanamido)thiazol-5-yl)acetic acid monoacetate;
(S)-methyl 6-(3-cyclopentyl-2-(2-oxo-5-(trifluoromethyl)pyridin-1(2H)-yl)propanamido)nicotinate;
(S)-6-(3-cyclopentyl-2-(2-oxo-5-(trifluoromethyl)pyridin-1(2H)-yl)propanamido)nicotinic acid mono acetate; and
(S)-diethyl (5-(3-cyclopentyl-2-(2-oxo-5-(trifluoromethyl)pyridin-1(2H)-yl)propanamido)-pyrazin-2-yl)methylphosphonate;
(S)-3-cyclopentyl-N-(5-(hydroxymethyl)pyridin-2-yl)-2-(2-oxo-5-(trifluoromethyl)pyridin-1(2H)-yl)propanamide;
(S)-6-(3-cyclopentyl-2-(2-oxo-4-(trifluoromethyl)pyridin-1(2H)-yl)propanamido)nicotinic acid; and
(S)-6-(3-cyclohexyl-2-(2-oxo-4-(trifluoromethyl)pyridin-1(2H)-yl)propanamido)nicotinic acid;
or a pharmaceutically acceptable salt thereof.

9. The compound of claim 6 wherein
X is nitrogen; and
R⁶ is methyl, ethyl, methoxy, CF₃, methoxy, ethoxy, halo, —CH₂P(O)(OR⁷)(OR⁸), —C(O)OR⁷, —CH₂C(O)OR⁷, or benzyl, where R⁷ and R⁸ are each independently H, methyl, or ethyl;
or a pharmaceutically acceptable salt thereof.

10. A compound selected from the group consisting of
(S)-3-cyclopentyl-N-(5-methylpyrazin-2-yl)-2-(6-oxo-4-(trifluoromethyl)pyrimidin-1(6H)-yl)propanamide;
(S)-3-cyclopentyl-N-(5-methylpyridin-2-yl)-2-(6-oxo-4-(trifluoromethyl)pyrimidin-1(6H)-yl)propanamide;
(S)-3-cyclopentyl-N-(1-methyl-1H-pyrazol-3-yl)-2-(6-oxo-4-(trifluoromethyl)pyrimidin-1(6H)-yl)propanamide;
(S)-3-cyclopentyl-N-(5-(hydroxymethyl)pyridin-2-yl)-2-(2-oxo-5-(trifluoromethyl)pyridin-1(2H)-yl)propanamide;
(S)-6-(3-cyclopentyl-2-(2-oxo-4-(trifluoromethyl)pyridin-1(2H)-yl)propanamido)nicotinic acid; and
(S)-6-(3-cyclohexyl-2-(2-oxo-4-(trifluoromethyl)pyridin-1(2H)-yl)propanamido)nicotinic acid;
(S)-6-(3-cyclopentyl-2-(2-oxo-5-(trifluoromethyl)pyridin-1(2H)-yl)propanamido)nicotinic acid;
or a pharmaceutically acceptable salt thereof.

11. A pharmaceutical composition comprising a compound of any one of claims 1 to 10; and a pharmaceutically acceptable excipient, diluent, or carrier.

12. The composition of claim 11 wherein said compound, or a pharmaceutically acceptable salt thereof, is present in a therapeutically effective amount.

13. A method for treating or delaying the progression or onset of Type 2 diabetes and diabetes-related disorders in mammals comprising the step of administering to a mammal in need of such treatment a therapeutically effective amount of a compound of any one of claims 1 to 10.

14. A method for treating or delaying the progression or onset of Type 2 diabetes and diabetes-related disorders in mammals comprising the step of administering to a mammal in need of such treatment a pharmaceutical composition of any one of claims 11 to 12.

15. A method of reducing the level of blood glucose in a mammal, comprising administering to said mammal in need of such blood glucose reduction which method comprises administering to said mammal a therapeutically effective amount of a compound of claim 1, or a pharmaceutically acceptable salt thereof, wherein said mammal is human.
